SEQUENCE LISTING (11 sequences)

SEQ ID NO: 1 - protein, 9 aa, Homo sapiens
YPHFMPTNL

SEQ ID NO: 2 - protein, 10 aa, Homo sapiens
SGPSNTPPEI

SEQ ID NO: 3 - protein, 9 aa, Homo sapiens
FAPGNYPAL

SEQ ID NO: 4 - protein, 9 aa, Homo sapiens
SYIPSAEKI

SEQ ID NO: 5 - protein, 9 aa, Homo sapiens
EEGAIVGEI

SEQ ID NO: 6 - protein, 9 aa, Homo sapiens
RPQASGVYM

SEQ ID NO: 7 - protein, 10 aa, Artificial Sequence
  Synthetic peptide
SKKKKKKKKC

SEQ ID NO: 8 - protein, 9 aa, Escherichia coli
TPHPARIGL

SEQ ID NO: 9 - protein, 9 aa, Escherichia coli
DAPIYTNVT

SEQ ID NO: 10 - protein, 12 aa, Hepatitis B virus
IPQSLDSWWTSL

SEQ ID NO: 11 - protein, 10 aa, Artificial Sequence
  Synthetic peptide
KSKKKKKKKK

(19) United States
(12) Patent Application Publication
(10) Pub. No.: US 2010/0129385 A1
(43) Pub. Date: May 27, 2010

(54) IMMUNOGENIC MOLECULES

(76) Inventors: David C. Jackson, North Balwyn (AU); Weiguang Zeng, North Melbourne (AU)

Correspondence Address: KNOBBE MARTENS OLSON & BEAR LLP, 2040 MAIN STREET, FOURTEENTH FLOOR, IRVINE, CA, 92614, US

(21) Appl. No.: 11/815,847

(22) PCT Filed: Feb. 8, 2006
(86) PCT No.: PCT/AU06/00162

§ 371 Date: Oct. 8, 2007

(30) Foreign Application Priority Data
Feb. 8, 2005 (AU) 2005900571

(51) Int. Cl. A61K 39/00

(52) U.S. Cl. 424/185.1; 424/193.1

(57) ABSTRACT

The present invention relates generally to the field of immunology and more particularly to molecules capable of stimulating a cellular immune response. More particularly, the present invention provides self-adjuvanting immunogenic molecules capable of stimulating an immune response to epitopes of a polypeptide irrespective of a subjects HLA type. The present invention further contemplates methods for the production and use of the self-adjuvanting immunogenic molecules and compositions comprising same useful in the vaccination of subjects against specific polypeptides.

### BACKGROUND OF THE INVENTION

1. Field of the Invention

The present invention relates generally to the field of immunology and more particularly to molecules capable of stimulating a cellular immune response. More particularly, the present invention provides self-adjuvanting immunogenic molecules capable of stimulating an immune response to epitopes of a polypeptide irrespective of a subjects HLA type. The present invention further contemplates methods for the production and use of the self-adjuvanting immunogenic molecules and compositions comprising same useful in the vaccination of subjects against specific polypeptides.

2. Description of the Prior Art

The reference to any prior art in this specification is not, and should not be taken as, an acknowledgment or any form of suggestion that that prior art forms part of the common general knowledge in Australia.

Immunotherapy and vaccination are used in the prophylaxis or treatment of a wide range of disorders, such as infectious diseases and certain tumors. However, the application and success of such treatments are limited in part by the need to stimulate a multi-facetted immune response. For example, in order to generate and maintain a cytotoxic T lymphocyte (CTL) response to a specific antigen, it requires the presence of a T helper (Th) response to the same antigen. Stimulation of a Th response inter alia induces the production of IL-2 from the cells, which in turn allows clonal expansion of a CTL directed to the same antigen.

T cells are capable of recognising peptide fragments which have been processed and are bound to major histocompatibility (MHC) molecules present on the surface of antigen presenting cells (APC). The MHC complex comprise two sets of highly polymorphic cell-surface molecules, termed MHC class I and MHC class II. MHC class I molecules bind to peptides produced by the degradation of molecules which are within the APC. MHC class I/peptide complexes present to CD8⁺ T cells which recognise a specific combination of MHC class I molecule and peptide. MHC class II molecules bind to peptides produced following breakdown of proteins which have been endocytosed by the APC. MHC class II/peptide complexes present to CD4⁺ Th cells which recognise a specific combination of MHC II molecule and peptide. Typically, a CD4⁺ Th response to a specific antigen is stimulated by a different peptide than stimulates the antigen specific CD8⁺ CTL response.

The peptide binding cleft on the MHC molecule is formed during the folding of the MHC. Binding pockets within the clefts are able to accommodate different peptides depending on the haplotype. The human class I family contains three principle class I loci, called HLA-A, HLA-B and HLA-C. There are also HLA-E, -F, -G and -H, however, these genes are far less polymorphic that the HLA-A, -B and -C loci. The human class II contains three major loci, designated DR, DQ and DP. Both the class I and II loci can then be further divided into a infinite number of sub-classes.

MHC molecules are co-dominantly expressed. This means that in one individual, all of the principal gene loci are expressed from both maternal and paternal chromosomes. As there are three class I loci, and as each of the loci is highly polymorphic, most individuals will have six different class I molecules. Each MHC molecule will have a slightly different shape and therefore, will present a different antigenic peptide. A similar process is applicable to MHC class II. At first sight, it would appear that an APC could express 6 different class II molecules, however, this is likely an underestimate due to hybrid class II molecules. Accordingly, it can be appreciated that there will be a high level of variability between individuals in relation to their HLA type.

Each MHC molecule is capable of binding to a different peptide fragment from a protein, and once bound, a specific CD8⁺ CTL or CD4⁺ Th is then capable of recognising this peptide, but only in the context of a specific HLA type. Accordingly, an HIV-specific peptide which is capable of stimulating a CTL response in a person who is HLA-A2, may not be capable of stimulating a response in a person who does not contain cells expressing A2 on their surfaces.

Such diversity in the immune system is a severe hindrance when trying to develop a vaccine or therapy against a specific pathogen or tumour. Often, only a single peptide capable of eliciting a CTL response is administered. Such vaccination results not only in a highly restricted immune response, but also does not provide peptides which are likely to stimulate Th responses and thus provide the requisite “help”. Furthermore, the use of such strategies in the vaccination or therapy of viruses has typically resulted in an evolutionary switch in the viral genome, where such a response is rendered ineffectual. Delivery strategies are also limited as full-length proteins containing CTL epitopes do not effectively enter the MHC class I processing pathway.

There is a need therefore to develop immunogenic molecules capable of stimulating an immune response irrespective of a subject's HLA type.

### SUMMARY OF THE INVENTION

Throughout the specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention is directed to polypeptides capable of eliciting an immune response in a subject irrespective of the subject's HLA type. More particularly, the present invention provides self-adjuvanting immunogenic molecules comprising naturally occurring or recombinant polypeptides which are conjugated to at least one lipid or fatty acid moiety, wherein the self-adjuvanting immunogenic molecule is capable of stimulating an immune response specific for epitopes within the naturally occurring or recombinant polypeptide. The self-adjuvanting immunogenic molecule of the present invention is capable of eliciting an immune response specifically against the naturally occurring or recombinant polypeptide, wherein in the immune response is characterized by the presence of CD8⁺ CTL and CD4⁺ T helper and or B cells all specific for the polypeptide.

Accordingly, one aspect of the present invention is directed to a self-adjuvanting immunogenic molecule comprising a naturally occurring or recombinant polypeptide conjugated to one or more lipid or fatty acid moieties, wherein the polypeptide comprises an amino acid sequence which contains at least one CTL epitope and one T-helper epitope or one CTL epitope and one B cell epitope or one T helper epitope and one B cell epitope or one CTL epitope, one T helper epitope and one B cell epitope, wherein the epitopes are specific for the polypeptide and wherein the self-adjuvanting immunogenic molecule elicits an immune response in a subject irrespective of the subject's HLA type.

In a particular aspect, the polypeptide of the present invention contains at least one CTL and one T helper epitope which are all capable of eliciting an immune response specific for the naturally occurring or recombinant polypeptide.

In a related aspect of the present invention, the polypeptide contains one CTL epitope and one B cell epitope which are all capable of eliciting an immune response specific for the naturally occurring or recombinant polypeptide.

In a further aspect of the present invention, the polypeptide contains one T helper epitope and one B cell epitope which are all capable of eliciting an immune response specific for the naturally occurring or recombinant polypeptide.

In a preferred aspect, the polypeptide contains at least one CTL epitope and at least one T helper epitope and at least one B cell epitope which are all capable of eliciting an immune response specific for the naturally occurring or recombinant polypeptide.

In a particular embodiment, the present invention contemplates a self-adjuvanting immunogenic molecule comprising a naturally occurring or recombinant polypeptide conjugated to one or more lipid or fatty acid moieties, wherein the polypeptide comprises an amino acid sequence which contains at least one CTL epitope and one T-helper epitope and one B cell epitope, wherein the epitopes are specific for the polypeptide and wherein the self-adjuvanting immunogenic molecule elicits an immune response in a subject irrespective of the subject's HLA type.

In addition, the present invention provides a self-adjuvanting immunogenic molecule comprising a naturally occurring or recombinant polypeptide conjugated to one or more lipid or fatty acid moieties, wherein the self-adjuvanting immunogenic molecule elicits an immune response in a subject irrespective of the subject's HLA type.

The lipid or fatty acid moiety may be conjugated to any amino acid residue within the polypeptide backbone or to a post translationally added chemical entity such as a carbohydrate moiety. In a preferred embodiment, the lipid or fatty acid moiety is conjugated to a side-chain of the amino acid or the N-terminus of the polypeptide. Conveniently, the conjugation of the lipid or fatty acid moiety to the polypeptide does not significantly alter the natural folding of the polypeptide and therefore allows the presentation of both linear and conformational epitopes.

In another aspect, the present invention provides a method for generating a self-adjuvanting immunogenic molecule said method comprising selecting or preparing a naturally occurring or recombinant polypeptide comprising an amino acid sequence which contains at least one CTL epitope and one T-helper epitope or one CTL epitope and one B cell epitope or one T helper epitope and one B cell epitope or one CTL epitope, one T helper epitope and one B cell epitope and conjugating at least one lipid or fatty acid moiety to any amino acid residue within the polypeptide or to a post translationally added chemical moiety on the naturally occurring or recombinant polypeptide, wherein the self-adjuvanting immunogenic molecule elicits an immune response in a subject irrespective of the subject's HLA type.

The present invention provides compositions comprising the self-adjuvanting immunogenic molecules and to the use of the compositions or self-adjuvanting immunogenic molecules in the manufacture of a medicament for treating or preventing cancer or infections by pathogens.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of the sequence identifiers is provided in Table 1. A sequence listing is provided after the claims.

A summary of the sequence identifiers used herein are shown in Table 1.

TABLE 1
Sequence Identifiers

| Sequence Identifier | Sequence |
|---|---|
| SEQ ID NO: 1 | CTL epitope for IFNγ |
| SEQ ID NO: 2 | CTL epitope for IFNγ |
| SEQ ID NO: 3 | CTL epitope for IFNγ |
| SEQ ID NO: 4 | CTL epitope for IFNγ |
| SEQ ID NO: 5 | CTL epitope for IFNγ |
| SEQ ID NO: 6 | CTL epitope for IFNγ |
| SEQ ID NO: 7 | spacer sequence for lipid moiety |

### BRIEF DESCRIPTION OF THE FIGURES

FIG. 1 is a diagrammatic representation showing schematic diagram of water-soluble Pam2Cys-based lipid moieties which have 8 lysine residues as spacer and which can be used as modules to lipidate protein in aqueous solution.

FIG. 2 is a diagrammatic representation showing schematic diagram of water-soluble Pam2Cys-based lipid moieties which have polyethylene glycol as spacer and which can be used as modules to couple to protein in aqueous solution.

FIG. 3 is a diagrammatic representation showing schematic diagram of various lipidated hen eggwhite lysozyme species and the various chemical linkages used.

FIG. 4 is a graphical representation showing anti-insulin antibody responses elicited by insulin and lipidated insulin in BALB/c mice. Mice were inoculated by the subcutaneous route at weeks 0 and 4 with insulin emulsified in complete Freund's adjuvant for the first dose and in incomplete Freund's adjuvant for the second dose. In each case the dose of antigen used was 10 nmole. In the case of lipidated insulin two doses were again administered but in this case in PBS. Sera were prepared from blood samples taken at weeks 4, 5 and 6 and anti-insulin antibody titres were then determined by ELISA. 1°, 2° and 3° represent the titres of antibody obtained at weeks 4, 5 and 6 respectively. Pam2Cys₂-insulin refers to insulin in which 2 copies of the lipid moiety Pam2Cys were incorporated into each molecule of insulin and Pam2Cys3-insulin refers to insulin in which 3 copies of the lipid moiety Pam2Cys were incorporated into each molecule of insulin. Pam2Cys-Ser-Lys₈-Cys is Pam2Cys to which are attached a serine residue, 8 lysine residues and a C-terminal cysteine residue. This structure represents the soluble form of Pam2Cys used to couple to the insulin molecules.

FIG. 5 is a graphical representation showing antibody responses elicited in C57BL6 by lipidated hen eggwhite lysozyme (Pam2Cys-HEL), HEL co-admixed with the lipid moiety Pam2CysSer(Lys)8Cys, HEL in Freund's adjuvant, HEL alone, Freund's adjuvant alone. Mice received two doses (30 μg each dose) of HEL at weeks 0 and 4 by the sub-cutaneous route and were bled at weeks 4 and 6. Anti-HEL antibody responses were determined in sera obtained at week 4 (1°) and week 6 (2°) by ELISAs.

FIG. 6 is a graphical representation showing antibody responses elicited in C57BL6 and BALB/c mice by lipidated hen eggwhite lysozyme ((Pam2Cys-HEL), HEL alone or HEL in complete Freund's adjuvant. Mice received two doses (25 μg) of HEL at weeks 0 and 3 by the sub-cutaneous route and were bled at weeks 3 and 5. Anti-HEL antibody responses were determined in sera obtained at week 3 (1°) and week 5 (2°) by ELISA.

FIG. 7 is a graphical representation showing anti-HEL antibody responses in C57BL6 mice inoculated with various forms of lipidated HEL. HEL containing a single copy of Pam2Cys (pam2Cys₁) or two copies of Pam2Cys (Pam2Cys₂) attached to the protein by either thioether or disulphide linkage were inoculated into C57BL6 mice. A separate group of mice were inoculated with HEL conjugated with 2 copies of Pam2Cys in a branched configuration (see FIG. 3). Control groups of animals were inoculated with HEL emulsified in complete Freund's adjuvant (CFA) or with HEL co-admixed with Pam2CysSer-Lys₈-Cys in the ratio 1:4. Mice received two doses of 25 μg of protein at weeks 0 and 3 by the sub-cutaneous route and were bled at weeks 3 and 5. Sera were prepared and anti-HEL antibody responses determined by ELISA.

FIG. 8 is a graphical representation showing anti-HEL antibody responses elicited in C57BL/6 and GK 1.5 mice by lipidated HEL (Pam2Cys-HEL) administered in saline, HEL administered in Freund's adjuvant or HEL in saline. Mice received two doses (25 μg each dose) antigen at weeks 0 and 4 and were bled at weeks 4 and 6. Sera were prepared from blood and the anti-HEL antibody responses were determined by ELISA.

FIG. 9 is a graphical representation showing anti-HEL antibody responses induced in C57BL6 mice by lipidated HEL (Pam2Cys₁-HEL) manufactured using disulphide chemistry (FIG. 3), HEL emulsified in Freund's adjuvant (HEL/CFA) or HEL administered in alum (HEL/alum). Mice received two doses (25 μg each dose) of antigen on days 0 and 21 and were bled on days 21 and 31. Sera were prepared and anti-HEL antibody responses were determined by ELISA. A significant secondary anti-HEL antibody response was obtained when lipidated HEL was administered compared to those when the non-lipidated HEL was administered in ALUM or in the presence of Freund's adjuvant.

FIG. 10 is a graphical representation showing antibody isotypes induced in BALM mice by lipidated HEL (Pam2Cys-HEL) in saline or HEL administered in complete Freund's adjuvant. Mice were inoculated sub-cutaneously with 2 doses (30 μg each dose) Pam2Cys-HEL or HEL emulsified in complete Freund's adjuvant (CFA) on days 0 and 28. Animals were bled 14 days following the second dose of antigen, sera prepared and the isotype of anti-HEL antibodies determined by ELISA.

FIG. 11 is a graphical representation showing antibody responses in C57BL/6 mice inoculated with ovalbumin (OVA). Animals received two doses of 30 μg of lipidated OVA (Pam2Cys-OVA), OVA emulsified in complete Freund's adjuvant (CFA) or OVA in saline administered sub-cutaneously on days 0 and 21. Mice were bled on days 21 (1°) and 31 (2°), sera prepared and anti-OVA antibody responses determined by ELISA.

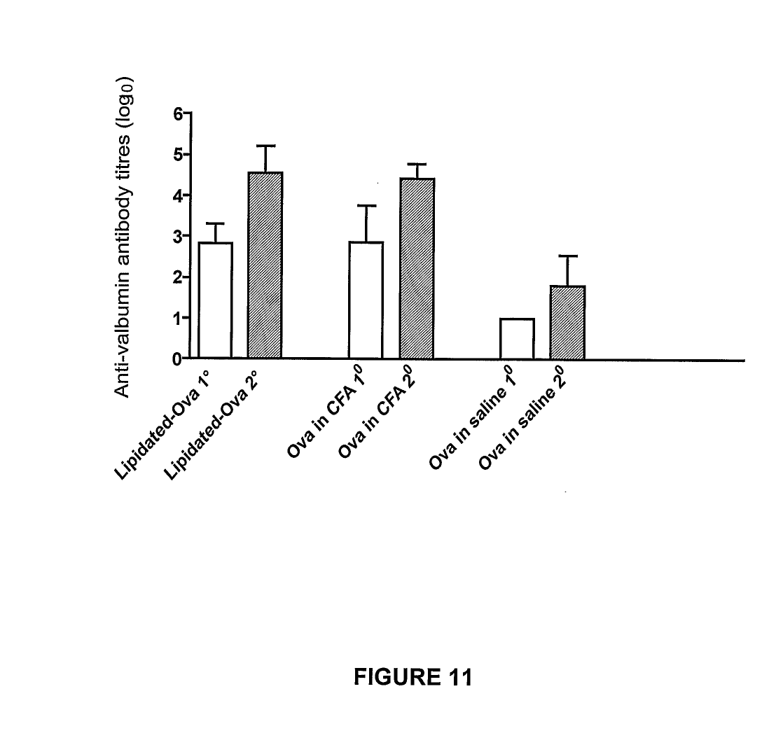

FIG. 12 is a graphical representation showing antibody isotypes elicited in C57BL6 mice following inoculation with lipidated OVA (Pam2Cys-OVA) or OVA emulsified in compete Freund's adjuvant on days 0 and 23. Mice were inoculated sub-cutaneously with two doses (30 μg each dose) of either Pam2Cys-OVA or OVA emulsified in complete Freund's adjuvant (CFA). Animals were bled on day 33, sera prepared and the isotype of anti-OVA antibodies determined by ELISA.

FIG. 13 is a graphical representation showing induction of CD8⁺ T cells by lipidated ovalbumin (OVA). C57BL/6 mice were inoculated subcutaneously with two doses (30 μg each) of either untreated ovalbumin in saline or Pam2Cys-OVA in saline on days 0 and 7. On Day 14 spleens were removed and splenocytes examined by intracellular cytokine staining for interferon-γ secretion following stimulation with the ovalumin CTL peptide epitope SIINFEKL or an irrelevant peptide for 4 hrs. IFN-γ was detected by flow analysis.

FIG. 14 is a graphical representation of CTL induction by lipidated polytopes. BALB/c and C57BL6 mice were inoculated sub-cutaneously (base of tail) with 9 nmoles (BALB/c mice) or 5 nmoles (C57BL6 mice). Seven days later spleens were removed and IFNγ-ELISpot assays performed on the splenocytes in the presence or absence of the following CTL peptide epitopes: SYIPSAEKI (SEQ ID NO:4) which is H-2K^{d}-restricted and comes from the circumsporazoite protein of P. berghei or epitope SGPSNTPPEI (SEQ ID NO:2) which is H-2D^{b}-restricted and comes from adenovirus 5EIA. The results are shown in the left and right panels respectively.

### DETAILED DESCRIPTION OF THE PREFERRED EMBODIMENTS

The present invention employs molecules, and in particular naturally occurring or recombinant polypeptides conjugated to lipid or fatty acid moieties for use in stimulating immune responses specific for epitopes within naturally occurring or recombinant polypeptides. The response occurs in a subject irrespective of the subject's HLA type. Reference to an “immune response” includes either a cellular response or a humoral immune response or both. In a preferred aspect, the cellular immune response includes a cytotoxic T cell response and a T helper response or a cytotoxic T cell response and a B cell response or a T helper response and a B cell response or a cytotoxic T cell response and a T helper response and a B cell response.

Accordingly, one aspect of the present invention provides a self-adjuvanting immunogenic molecule comprising a naturally occurring or recombinant polypeptide associated with one or more lipids or fatty acid moieties, wherein the naturally occurring or recombinant polypeptide comprises an amino acid sequence which comprises at least one CTL epitope and one T-helper epitope or one CTL epitope and one B cell epitope or one T helper epitope and one B cell epitope or one CTL epitope and at least one T helper epitope and at least one B cell epitope, wherein the epitopes are specific for the polypeptide and wherein the self-adjuvanting immunogenic molecule is capable of stimulating an immune response to the epitopes on the polypeptide irrespectively of the HLA type of a subject.

As used herein, a “self-adjuvanting immunogenic molecule” refers to the ability of the naturally occurring or recombinant polypeptide to stimulate a cytotoxic T cell response and/or a T helper response and/or B cell response without the aid of an additional adjuvant.

As used herein, a “T helper epitope” can also be defined as a “Th epitope” or CD4⁺ T helper epitope” and includes any epitope capable of enhancing or stimulating a CD4⁺ T cell response when administered to a subject. Preferred T helper epitopes comprise at least about 6, 7, 8, 9, 10, 11, 12, 13, 14, 15, 16, 17, 18, 19, 20, 21, 22, 23, 24, 25, 26, 27, 28, 29 or 30 amino acids in length.

As used herein, a “CTL epitope” can also be defined as a “cytotoxic T cell epitope” or “CD8⁺ CTL epitope” and includes any epitope which is capable of enhancing or stimulating a CD8⁺ T cell response when administered to a subject. Preferred CTL epitopes comprise at least about 6, 7, 8, 9, 10, 11, 12, 13, 14, 15, 16, 17, 18, 19, 20, 21, 22, 23, 24, 25, 26, 27, 28, 29 or 30 amino acids in length.

As used herein, a “B cell epitope” is any epitope which is capable of eliciting the production of antibodies when administered to a subject. Preferably, the B cell epitope is capable of eliciting neutralizing antibodies, and more preferably, high titer neutralizing antibodies. Preferred B cell epitopes comprise at least about 4, 5, 6, 7, 8, 9, 10, 11, 12, 13, 14, 15, 16, 17, 18, 19, 20, 21, 22, 23, 24, 25, 26, 27, 28, 29 or 30 amino acids in length.

As used herein, the term “polypeptide” “is used in its conventional meaning, i.e., as a sequence of amino acids. The naturally occurring or recombinant polypeptides of the present invention, therefore, should be understood to also encompass peptides, oligopeptides and proteins. The protein may be glycosylated or unglycosylated (i.e. comprise a carbohydrate entity) and/or may contain a range of other molecules fused, linked, bound or otherwise associated to the protein such as amino acids, lipids, carbohydrates or other peptides, polypeptides or proteins. Reference hereinafter to a “protein” includes a protein comprising a sequence of amino acids as well as a protein associated with other molecules such as amino acids, lipids, carbohydrates or other peptides, polypeptides or proteins. Reference to a “carbohydrate entity” or a “glycosylated entity” includes a synthetically or naturally modified entity.

The polypeptides must be of a length which contains at least one CTL epitope and one T-helper epitope or one CTL epitope and one B cell epitope or one T helper epitope and one B cell epitope or one CTL epitope and one T helper epitope and one B cell epitope. As indicated above, the terms peptides, oligopeptides and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. Particular polypeptides of interest in the context of this invention are amino acid subsequences comprising epitopes, i.e., antigenic determinants substantially responsible for the immunogenic properties of a polypeptide and being capable of evoking an immune response in an HLA-independent manner.

The polypeptides of the invention are immunogenic, i.e., they are able to stimulate T-cells and/or B-cells from a subject specific for a target polypeptide without the additional of an adjuvant. Screening for immunogenic activity can be performed using techniques well known to the skilled artisan. For example, such screens can be performed using methods such as those described in Harlow and Lane, Antibodies: A Laboratory Manual, Cold Spring Harbor Laboratory, Cold Spring Harbor, N.Y., USA, 1988. In one illustrative example, a polypeptide may be immobilized on a solid support and contacted with patient sera to allow binding of antibodies within the sera to the immobilized polypeptide. Unbound sera may then be removed and bound antibodies detected using, for example, I¹²⁵ labeled Protein A.

An “immunogenic portion,” or “epitope” as used herein, is a fragment of an immunogenic polypeptide of the subject invention that itself is immunologically reactive (i.e., specifically binds) with the B-cells and/or T-cell surface antigen receptors that recognize the polypeptide. Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared using well-known techniques.

In one preferred embodiment, a self-adjuvanting immunogenic molecule of the present invention comprises a polypeptide a portion of which reacts with antisera and T-cells at a level that is not substantially less than the reactivity of the full-length polypeptide (e.g., in an ELISA and/or T-cell reactivity assay). Preferably, the level of immunogenic activity of the self-adjuvanting immunogenic molecule is at least about 50%, preferably at least about 70% and most preferably greater than about 90% of the immunogenicity for the full-length polypeptide. In some instances, preferred immunogenic portions will be identified that have a level of immunogenic activity greater than that of the corresponding full-length polypeptide, e.g., having greater than about 100% or 150% or more immunogenic activity.

The present invention, contemplates polypeptides comprising at least about 5, 10, 15, 20, 25, 50, or 100 contiguous amino acids, or more, including all intermediate lengths.

In order to express a recombinant polypeptide, the nucleotide sequences encoding the polypeptide, or functional equivalents, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook et al. (1989) Molecular Cloning, A Laboratory Manual, Cold Spring Harbor Press, Plainview, N.Y., and Ausubel et al. Current Protocols in Molecular Biology, John Wiley & Sons, New York. N.Y, 1989.

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” may also be referred to as “regulatory sequences”. These sequences present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, any of a number of expression vectors may be selected depending upon the use intended for the expressed polypeptide. For example, when large quantities are needed, for example for the induction of antibodies, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of .beta.-galactosidase so that a hybrid protein is produced; pIN vectors (Van Heeke et al. J Biol Chem 264:5503-5509, 1989); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. supra and Grant et al. Methods Enzymol 153:516-544, 1987.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu EMBO J 6:307-311, 1987. Alternatively, plant promoters such as the small subunit of RUBISCO or heat shock promoters may be used (Coruzzi et al. EMBO J. 3:1671-1680, 1984; Broglie et al. Science 224:838-843, 1984; Winter et al. Results Probl Cell Differ 17:85-105, 1991. These constructs can be introduced into plant cells by direct DNA transformation or pathogen-mediated transfection. Such techniques are described in a number of generally available reviews (see, for example, Hobbs or Murry. in McGraw Hill Yearbook of Science and Technology McGraw Hill, New York, N.Y.; pp. 191-196, 1992).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard et al. Proc Natl Acad Sci 91:3224-3227, 1994).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan et al. Proc Natl Acad Sci 81:3655-3659, 1984). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf et al. Results Probl Cell Differ 20:125-162, 1994).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, COS, HeLa, MDCK, HEK293, and WI38, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen. San Diego, Calif.) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on BOAC (immobilized metal ion affinity chromatography) as described in Porath et al. Prot Exp Purif 3:263-281, 1992 while the enterokinase cleavage site provides a means for purifying the desired polypeptide from the fusion protein. A discussion of vectors which contain fusion proteins is provided in Kroll et al. DNA Cell Biol 12:441-453, 1993.

T cells are considered to be specific for a polypeptide of the present invention if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al. Cancer Res 54:1065-1070, 1994. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a tumor polypeptide (100 ng/ml-100 μg/ml, preferably 200 ng/ml-25 μg/ml) for 3-7 days will typically result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ.) is indicative of T cell activation (see Coligan et al. Current Protocols in Immunology, vol. 1, Wiley Interscience (Greene 1998)). T cells that have been activated in response to a tumor polypeptide, polynucleotide or polypeptide-expressing APC may be CD4⁺ and/or CD8⁺. Tumor polypeptide-specific T cells may be expanded using standard techniques. Within preferred embodiments, the T cells are derived from a patient, a related donor or an unrelated donor, and are administered to the patient following stimulation and expansion.

For therapeutic purposes, CD4⁺ or CD8⁺ T cells that proliferate in response to a specific polypeptide can be expanded in number either in vitro or in vivo. Proliferation of such T cells in vitro may be accomplished in a variety of ways. For example, the T cells can be re-exposed to the polypeptide, or a short peptide corresponding to an immunogenic portion of such a polypeptide, with or without the addition of T cell growth factors, such as interleukin-2, and/or stimulator cells that synthesize a tumor polypeptide. Alternatively, one or more T cells that proliferate in the presence of the tumor polypeptide can be expanded in number by cloning. Methods for cloning cells are well known in the art, and include limiting dilution.

In a preferred aspect, the lipid or fatty acid moiety is conjugated to the polypeptide via an amino acid residue. The residue may be at any position within the polypeptide, including within the immunogenic epitopes themselves. Further, a lipid or fatty acid moiety may be conjugated to more than one residue within the polypeptide. In a preferred aspect, the amino acid residue is a lysine residue or cysteine residue or serine residue. The lipid or fatty acid moiety may also be bound to a post-translationally added chemical entity such as a carbohydrate.

Several different fatty acids are known for use in lipid moieties. Exemplary lipids or fatty acid moieties include, without being limited to, palmitoyl, myristoyl, stearoyl and decanoyl groups or, more generally, any C₂ to C₃₀ saturated, monounsaturated, or polyunsaturated fatty acyl group is thought to be useful.

An example of a specific fatty acid moiety the lipoamino acid N-palmitoyl-S-[2,3-bis(palmitoyloxy)propyl]cysteine, also known as Pam₃Cys or Pam₃Cys-OH (Wiesmuller et al. Hoppe Seylers Zur Physiol Chem 364:593, 1983) which is a synthetic version of the N-terminal moiety of Braun's lipoprotein that spans the inner and outer membranes of Gram negative bacteria. Pam₃Cys has the structure of Formula (I):

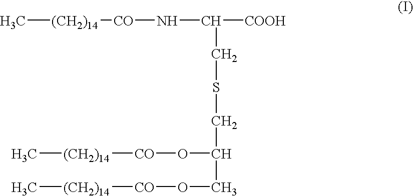

Pam₂Cys (also known as dipalmitoyl-5-glyceryl-cysteine or S-[2,3-bis(palmitoyloxy)propyl]cysteine, an analogue of Pam₃Cys, has been synthesised (Metzger. et al., J. Pept. Sci. 1:184, 1995) and been shown to correspond to the lipid moiety of MALP-2, a macrophage-activating lipopeptide isolated from mycoplasma (Sacht et al. Eur J Immunol 28:4207, 1998; Muhlradt et al. Infect Immun 66:4804, 1998; Muhlradt et al. J Exp Med 185:1951, 1997). Pam₂Cys has the structure of Formula (II):

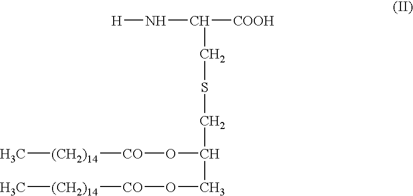

The lipid or fatty acid moiety conjugated to the self-adjuvanting immunogenic molecule of the present invention may be directly or indirectly attached to the polypeptide meaning that they are either juxtaposed in the self-adjuvanting immunogenic molecule (i.e. they are not separated by a spacer molecule) or separated by a spacer comprising one or more carbon-containing molecules, such as, for example, one or more amino acid residues. The polypeptide may be of any length. Preferably, it must be of a length that contains at least one CTL epitope and one T-helper epitope or one CTL epitope and one B cell epitope or one T helper epitope and one B cell epitope or one CTL epitope, one T helper epitope and one B cell epitope.

The lipid moiety is preferably a compound having a structure of general Formula (III):

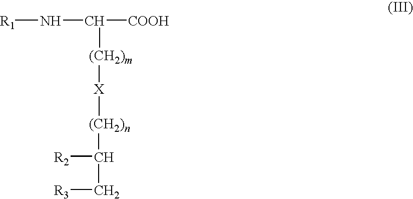

wherein:
(i) X is selected from the group consisting of sulfur, oxygen, disulfide (—S—S—), and methylene (—CH₂—), and amino (—NH—);
(ii) m is an integer being 1 or 2;
(iii) n is an integer from 0 to 5;
(iv) R₁ is selected from the group consisting of hydrogen, carbonyl (—CO—), and R′—CO-wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group;
(v) R₂ is selected from the group consisting of R′—CO—O—, R′—O—, R′—O—CO—, R′—NH—CO—, and R′—CO—NH—, wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group; and
(vi) R₃ is selected from the group consisting of R′—CO—O—, R′—O—, R′—O—CO—, R′—NH—CO—, and R′—CO—NH—, wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group
and wherein each of R₁, R₂ and R₃ is the same or different.

Depending upon the substituent, the lipid moiety of general Formula (III) may be a chiral molecule, wherein the carbon atoms directly or indirectly covalently bound to integers R₁ and R₂ are asymmetric dextrorotatory or levorotatory (i.e. an R or S) configuration.

Preferably, X is sulfur; m and n are both 1; R₁ is selected from the group consisting of hydrogen, and R′—CO—, wherein R′ is an alkyl group having 7 to 25 carbon atoms; and R₂ and R₃ are selected from the group consisting of R′—CO—O—, R′—O—, R′—O—CO—, R′—NH—CO—, and R′—CO—NH—, wherein R′ is an alkyl group having 7 to 25 carbon atoms.

Preferably, R′ is selected from the group consisting of: palmitoyl, myristoyl, stearyl and decanol. More preferably, R′ is palmitoyl.

Each integer R′ in said lipid moiety may be the same or different.

In a particularly preferred embodiment, X is sulfur; m and n are both 1; R₁ is hydrogen or R′—CO-wherein R′ is palmitoyl; and R₂ and R₃ are each R′—CO—O— wherein R′ is palmitoyl. These particularly preferred compounds are shown by Formula (I) and Formula (II) supra.

The lipid moiety can also have the following General Formula (IV):

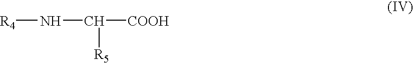

wherein:
(i) R₄ is selected from the group consisting of: (i) an alpha-acyl-fatty acid residue consisting of between about 7 and about 25 carbon atoms; (ii) an alpha-alkyl-beta-hydroxy-fatty acid residue; (iii) a beta-hydroxy ester of an alpha-alkyl-beta-hydroxy-fatty acid residue wherein the ester group is preferably a straight chain or branched chain comprising more than 8 carbon atoms; and (iv) a lipoamino acid residue; and
(ii) R₅ is hydrogen or the side chain of an amino acid residue.

Preferably, R₄ consists of between about 10 and about 20 carbon atoms, and more preferably between about 14 and about 18 carbon atoms.

Optionally, wherein R₄ is a lipoamino acid residue, the side-chain of the integers R₄ and R₅ can form a covalent linkage. For example, wherein R₄ comprises an amino acid selected from the group consisting of lysine, ornithine, glutamic acid, aspartic acid, a derivative of lysine, a derivative of ornithine, a derivative of glutamic acid, and a derivative of aspartic acid, then the side chain of that amino acid or derivative is covalently attached, by virtue of an amide or ester linkage, to R₅.

Preferably, the structure set forth in general Formula IV is a lipid moiety selected from the group consisting of: N,N′-diacyllysine; N,N′-diacylornithine; di(monoalkyl) amide or ester of glutamic acid; di(monoalkyl) amide or ester of aspartic acid; a N,O-diacyl derivative of serine, homoserine, or threonine; and a N,S-diacyl derivative of cysteine or homocysteine.

Amphipathic molecules, particularly those having a hydrophobicity not exceeding the hydrophobicity of Pam₃Cys (Formula (I)) are also preferred. The lipid moieties of Formula (I), Formula (II), Formula (III) or Formula (IV) are further modified during synthesis or post-synthetically, by the addition of one or more spacer molecules, preferably a spacer that comprises carbon, and more preferably one or more amino acid residues. These are conveniently added to the lipid structure via the terminal carboxy group in a conventional condensation, addition, substitution, or oxidation reaction. The effect of such spacer molecules is to separate the lipid moiety from the polypeptide moiety and increase immunogenicity of the lipopeptide product.

Serine dimers, trimers, tetramers, etc, are particularly preferred for this purpose.

Exemplary modified lipoamino acids produced according to this embodiment are presented as Formulae (V) and (VI), which are readily derived from Formulae (I) and (II), respectively by the addition of a serine homodimer. As exemplified herein, Pam₃Cys of Formula (I), or Pam₂Cys of Formula (II) is conveniently synthesized as the lipoamino acids Pam₃Cys-Ser-Ser of Formula (V), or Pam₂Cys-Ser-Ser of Formula (VI) for this purpose.

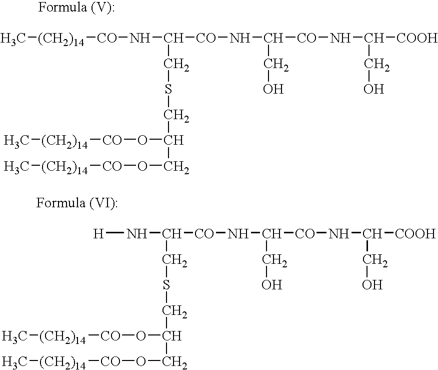

The lipid moiety is prepared by conventional synthetic means, such as, for example, the methods described in U.S. Pat. Nos. 5,700,910 and 6,024,964, or alternatively, the method described by Wiesmuller et al. 1983 supra, Zeng et al. J Pept. Sci 2:66, 1996; Jones et al. Xenobiotica 5:155, 1975; or Metzger et al. Int J Pept Protein Res 38:545, 1991). Those skilled in the art will be readily able to modify such methods to achieve the synthesis of a desired lipid for use conjugation to a polypeptide.

Other functional groups such as sulfhydryl, aminooxyacetyl, aldehyde may be introduced into the lipid moieties to enable the lipid moieties to couple to the naturally occurring or recombinant proteins more specifically.

Combinations of different lipids are also contemplated for use in the self-adjuvanting immunogenic molecules of the invention. For example, one or two myristoyl-containing lipids or lipoamino acids are attached via lysineresidues to the polypeptide moiety, optionally separated from the polypeptide by a spacer, with one or two palmitoyl-containing lipid or lipoamino acid molecules attached to carboxy terminal lysine amino acid residues. Other combinations are not excluded.

The lipid or fatty acid moiety may comprise any C₂ to C₃₀ saturated, monounsaturated, or polyunsaturated linear or branched fatty acyl group, and preferably a fatty acid group selected from the group consisting of: palmitoyl, myristoyl, stearoyl, lauroyl, octanoyl and decanol. Lipoamino acids are particularly preferred lipid moieties within the present context. As used herein, the term “lipoamino acid” refers to a molecule comprising one or two or three or more lipids covalently attached to an amino acid residue, such as, for example, cysteine or serine, lysine or an analog thereof. In a particularly preferred embodiment, the lipoamino acid comprises cysteine and optionally, one or two or more serine residues.

The structure of the lipid moiety is not essential to activity of the resulting self-adjuvanting immunogenic molecule and, as exemplified herein, palmitic acid and/or cholesterol and/or Pam₁Cys and/or Pam₂Cys and/or Pam₃Cys can be used. The present invention clearly contemplates a range of other lipid moieties for use in the self-adjuvanting immunogenic molecules without loss of immunogenicity. Accordingly, the present invention is not to be limited by the structure of the lipid moiety, unless specified otherwise, or the context requires otherwise.

Similarly, the present invention is not to be limited by a requirement for a single lipid moiety unless specified otherwise or the context requires otherwise. The addition of multiple lipid moieties to the naturally occurring or recombinant polypeptide, for example, to a position within an epitope or to a position between two epitopes is contemplated.

Polypeptides of the present invention are lipidated by methods well known in the art. Standard condensation, addition, substitution or oxidation The bifunctional linkers described in Pierce Catalogue and the methods therein may liberally be used here. As described in the examples, heterobifunctional linkers, MCS (N-Succinimidyl 6-maleimidocaproate) and SPDP (N-Succinimidyl 3-[2-pyridyldithio]propionate]) were used. In the case of using MCS as heterobifunctional linker, a cysteine residue was incorporated in the lipid moiety Pam2Cys-Ser-(Lys)8-Cys which was coupled to the MCS-modified protein by forming a thioether bond. Pam2Cys (Lys)8-Cys was also coupled to the SPDP modified protein by forming a disulfide bond.

Bromoacetyl or chloroacetyl group may also be introduced into the lipid moieties. These two functional groups can be coupled to the sulfhydryl groups existing or being introduced in the proteins by forming a thioether bond.

Another preferred method involves the incorporation of a serine residue in the N-terminal position of the polypeptide using recombinant or enzymatic or chemical method which is then oxidised to generate an aldehyde function. An aminooxy functional group incorporated in the lipid moiety will form an oxime bond to generate the self-adjuvanting lipid protein.

The other chemical ligation methods such as orthogonal ligation strategies (Tam et al. Biopolymers (Peptide Science) 51:311-332, 1999), native chemical ligation (Dawson et al. Science 266:243-247, 1994) expressed protein ligation (Muir et al. Proc Natl Acad Sci USA 95:6705-6710, 1998) may also be used to attached the lipid moiety to the polypeptide of the present invention.

As exemplified herein, highly self-adjuvanting immunogenic molecules capable of inducing CTL and/or Th and/or B cell responses are provided, wherein the self-adjuvanting immunogenic molecule in one aspect comprises Pam₃Cys of Formula (I), or Pam₂Cys of Formula (II) conjugated to the polypeptide.

The enhanced ability of the self-adjuvanting immunogenic molecules of the invention to elicit an immune response is reflected by their ability to upregulate the surface expression of MHC class II molecules on immature dendritic cells (DC), particularly D1 cells. Preferably, the self-adjuvanting immunogenic molecules are soluble, most preferably, highly soluble.

In one aspect, the present invention discloses the addition of multiple lipid or fatty acid moieties to the polypeptide.

The positioning of the lipid or fatty acid moiety should be selected such that the association of the lipid or fatty acid moiety does not interfere with the CTL, T helper or B cell epitope in such a way as to limit their ability to elicit an immune response. For example, depending on the selection of lipid or fatty acid moiety, the attachment within an epitope may sterically hinder the presentation of the epitope.

Preferably, the lipid or fatty acid moiety is associated with the polypeptide in a manner which does not alter the 3 dimensional structure of the protein. The present invention contemplates the presentation of linear epitopes as well as non-linear or “discontinuous” (conformational) epitopes. Conformational epitopes consist of amino acid residues that occur separated from each other within the primary, one-dimensional protein sequence, but that are within each other's proximity and accessible for antibodies on the surface of the folded, three-dimensional allergenic protein.

In additional embodiments, the present invention concerns formulation of one or more of the self-adjuvanting immunogenic molecules disclosed herein in pharmaceutically-acceptable carriers for administration to a subject either alone, or in combination with one or more other modalities of therapy.

It will be understood that, if desired, a composition as disclosed herein may be administered in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. The compositions may thus be delivered along with various other agents as required in the particular instance. Such compositions may be purified from host cells or other biological sources, or alternatively may be chemically synthesized as described herein.

Therefore, in another aspect of the present invention, pharmaceutical compositions are provided comprising one or more of the self-adjuvanting immunogenic molecules described herein in combination with a physiologically acceptable carrier. In certain preferred embodiments, the pharmaceutical compositions of the invention comprise self-adjuvanting immunogenic molecules of the invention for use in prophylactic and therapeutic vaccine applications. Vaccine preparation is generally described in, for example, Powell and Newman, eds., “Vaccine Design (the subunit and adjuvant approach)” Plenum Press (NY, 1995). Generally, such compositions will comprise one or more self-adjuvanting immunogenic molecules of the present invention in combination with one or more immunostimulants.

The self-adjuvanting immunogenic molecule is conveniently formulated in a pharmaceutically acceptable excipient or diluent, such as, for example, an aqueous solvent, non-aqueous solvent, non-toxic excipient, such as a salt, preservative, buffer and the like. Examples of non-aqueous solvents are propylene glycol, polyethylene glycol, vegetable oil and injectable organic esters such as ethyloleate. Aqueous solvents include water, alcoholic/aqueous solutions, saline solutions, parenteral vehicles such as sodium chloride, Ringer's dextrose, etc. Preservatives include antimicrobial, anti-oxidants, cheating agents and inert gases. The pH and exact concentration of the various components the pharmaceutical composition are adjusted according to routine skills in the art.

The addition of an extrinsic adjuvant to the self-adjuvanting immunogenic molecule formulation, although generally not required, is also encompassed by the invention. Such extrinsic adjuvants include all acceptable immunostimulatory compounds such as, for example, a cytokine, toxin, or synthetic composition. Exemplary adjuvants include IL-1, IL-2, BCG, aluminum hydroxide, N-acetyl-muramyl-L-threonyl-D-isoglutamine(thur-MDP), N-acetyl-nor-muramyl-L-alanyl-D-isoglutamine(CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine(CGP)1983A, referred to as MTP-PE), lipid A, MPL and RIBI, which contains three components extracted from bacteria, monophosphoryl lipid A, trehalose dimycolate and cell wall skeleton (MPL+TDM+CWS) in a 2% squalene/Tween 80 emulsion.

It may be desirable to co-administer biologic response modifiers (BRM) with the self-adjuvanting immunogenic molecule, to down regulate suppressor T cell activity. Exemplary BRM's include, but are not limited to, Cimetidine (CIM; 1200 mg/d) (Smith/Kline, PA, USA); Indomethacin (IND; 150 mg/d) (Lederle, NJ, USA); or low-dose Cyclophosphamide (CYP; 75,150 or 300 mg/m.²) (Johnson/Mead, NJ, USA).

The self-adjuvanting immunogenic molecules of the present invention are capable of eliciting a T cell and/or a B cell response either in vivo or ex vivo. More particularly, the self-adjuvanting immunogenic molecules of the present invention enhance CTL memory responses against the CTL epitope moiety when administered to an animal subject, without any requirement for an adjuvant to achieve a similar level of CTL activation. In addition, the self-adjuvanting immunogenic molecules of the present invention enhance maturation of dendritic cells and other biological effects including induction of IFN-γ producing CD8⁺ cells as well as viral, bacterial and tumour cell clearance.

Accordingly, a further aspect of the invention provides a method of enhancing cell mediated immunity against the polypeptide from which the T cell and/or B cell epitope is derived in a subject comprising administering the self-adjuvanting immunogenic molecule of the invention or a derivative or a functionally equivalent variant of said self-adjuvanting immunogenic molecule or a vaccine composition comprising said self-adjuvanting immunogenic molecule or variant or derivative for a time and under conditions sufficient to activate a CTL and/or a CTL precursor and/or Th and/or B cell of the subject.

Preferably, the self-adjuvanting immunogenic molecule or vaccine is administered prophylactically to a subject not harboring a latent or active infection by a parasite, bacterium or virus or suffering from a cancer or the self-adjuvanting immunogenic molecule is administered therapeutical to a subject harboring a latent or active infection by a parasite, bacteria or virus or suffering from a cancer. In the present context, the term “activate” means that the ability of a T cell to recognize and lyse a cell harboring an antigen from which the T cell epitope is derived is enhanced, or that the ability of a T cell to recognize a T cell epitope of said antigen is enhanced, either transiently or in a sustained manner. The term “activate” shall also be taken to include a reactivation of a T cell population following activation of a latent infection by a parasite or bacteria or virus, or following re-infection with a parasite or bacteria or virus, or following immunization of a previously-infected subject with a self-adjuvanting immunogenic molecule or composition of the invention.

Those skilled in the art are aware that optimum T cell activation requires cognate recognition of antigen/MHC by the T cell receptor (TcR), and a co-stimulation involving the ligation of a variety of cell surface molecules on the T cell with those on an antigen presenting cell (APC). The costimulatory interactions CD28/B7, CD40L/CD40 and OX40/OX40L are preferred, but not essential for T cell activation. Other costimulation pathways may operate.

For determining the activation of a CTL or precursor CTL or the level of epitope-specific activity, standard methods for assaying the number of CD8⁺ T cells in a specimen can be used. Preferred assay formats include a cytotoxicity assay, such as for example the standard chromium release assay, the assay for IFN-γ production, such as, for example, the ELISPOT assay. These assay formats are described in detail in the accompanying examples.

MHC class 1 Tetramer assays can also be utilized, particularly for CTL epitope-specific quantitation of CD8⁺ T cells (Altman et al. Science 274:94-96, 1996; Ogg et al. Curr Opin Immunol 10:393-396, 1998). To produce tetramers, the carboxyl terminus of an MHC molecule, such as, for example, the HLA A2 heavy chain, is associated with a specific peptide epitope or polyepitope, and treated so as to form a tetramer complex having bound thereto a suitable reporter molecule, preferably a fluorochrome such as, for example, fluoroscein isothiocyanate (FITC), phycoerythrin, phycocyanin or allophycocyanin. Tetramer formation is achieved, for example, by producing the MHC-peptide fusion protein as a biotinylated molecule and then mixing the biotinylated MHC-peptide with deglycosylated avidin that has been labeled with a fluorophore, at a molar ratio of 4:1. The Tetramers produced bind to a distinct set of CD8⁺ T cell receptors (TcRs) on a subset of CD8⁺ T cells derived from the subject (eg in whole blood or a PBMC sample), to which the peptide is HLA restricted. There is no requirement for in vitro T cell activation or expansion. Following binding, and washing of the T cells to remove unbound or non-specifically bound Tetramer, the number of CD8⁺ cells binding specifically to the HLA-peptide Tetramer is readily quantified by standard flow cytometry methods, such as, for example, using a FACSCalibur Flow cytometer (Becton Dickinson). The Tetramers can also be attached to paramagnetic particles or magnetic beads to facilitate removal of non-specifically bound reporter and cell sorting. Such particles are readily available from commercial sources (e.g. Beckman Coulter, Inc., San Diego, Calif., USA) Tetramer staining does not kill the labeled cells; therefore cell integrity is maintained for further analysis. MHC Tetramers enable the accurate quantitative analyses of specific cellular immune responses, even for extremely rare events that occur at less than 1% of CD8⁺ T cells (Bodinier et al. Nature Med 6:707-710, 2000; Ogg et al. Curr Opin Immunol 10:393-396, 1998).

The total number of CD8⁺ cells in a sample can also be determined readily, such as, for example, by incubating the sample with a monoclonal antibody against CD8 conjugated to a different reporter molecule to that used for detecting the Tetramer. Such antibodies are readily available (eg. Becton Dickinson). The relative intensities of the signals from the two reporter molecules used allows quantification of both the total number of CD8⁺ cells and Tetramer-bound T cells and a determination of the proportion of total T cells bound to the Tetramer.

Because CD4⁺ T-helper cells function in cell mediated immunity (CMI) as producers of cytokines, such as, for example IL-2, to facilitate the expansion of CD8⁺ T cells or to interact with the APC thereby rendering it more competent to activate CD8⁺ T cells, cytokine production is an indirect measure of T cell activation. Accordingly, cytokine assays can also be used to determine the activation of a CTL or precursor CTL or the level of cell mediated immunity in a human subject. In such assays, a cytokine such as, for example, IL-2, is detected or production of a cytokine is determined as an indicator of the level of epitope-specific reactive T cells.

Preferably, the cytokine assay format used for determining the level of a cytokine or cytokine production is essentially as described by Petrovsky et al. J Immunol Methods 186: 37-46, 1995, which assay reference is incorporated herein.

Preferably, the cytokine assay is performed on whole blood or PBMC or buffy coat.

Preferably, the self-adjuvanting immunogenic molecule or derivative or variant or vaccine composition is administered for a time and under conditions sufficient to elicit or enhance the expansion of T cells and/or B cells.

Still more preferably, the self-adjuvanting immunogenic molecule or derivative or variant or vaccine composition is administered for a time and under conditions sufficient for CMI to be enhanced in the subject.

By “CMI” is meant that the activated and clonally expanded CTLs are MHC— restricted and specific for a CTL epitope. CTLs are classified based on antigen specificity and MHC restriction, (i.e., non-specific CTLs and antigen-specific, MHC— restricted CTLS). Non-specific CTLs are composed of various cell types, including NK cells and can function very early in the immune response to decrease pathogen load, while antigen-specific responses are still being established. In contrast, MHC-restricted CTLs achieve optimal activity later than non-specific CTL, generally before antibody production. Antigen-specific CTLs inhibit or reduce the spread of a pathogen and preferably terminate infection.

CTL activation, clonal expansion, or CMI can be induced systemically or compartmentally localized. In the case of compartmentally localized effects, it is preferred to utilize a vaccine composition suitably formulated for administration to that compartment. On the other hand, there are no such stringent requirements for inducing CTL activation, expansion or CMI systemically in the subject.

The effective amount of self-adjuvanting immunogenic molecule to be administered, either solus or in a vaccine composition to elicit T cell and B cell activation, clonal expansion or CMI will vary, depending upon the nature of the immunogenic epitope, the route of administration, the weight, age, sex, or general health of the subject immunized, and the nature of the immune response sought. All such variables are empirically determined by art-recognized means.

The self-adjuvanting immunogenic molecule, optionally formulated with any suitable or desired carrier, adjuvant, BRM, or pharmaceutically acceptable excipient, is conveniently administered in the form of an injectable composition. Injection may be intranasal, intramuscular, sub-cutaneous, intravenous, intradermal, intraperitoneal, or by other known route. For intravenous injection, it is desirable to include one or more fluid and nutrient replenishers.

The optimum dose to be administered and the preferred route for administration are established using animal models, such as, for example, by injecting a mouse, rat, rabbit, guinea pig, dog, horse, cow, goat or pig, with a formulation comprising the self-adjuvanting immunogenic molecule, and then monitoring the immune response using any conventional assay.

The use of HLA A2/K^{b} transgenic mice carrying a chimeric human-mouse Class I MHC locus composed of the α1 and α2 domains of the human HLA A*0201 allele and the α3 domain of the mouse H-2K^{b} Class I molecules (Vitiello et al. J Exp Med 173:1007, 1991) is particularly preferred for testing responses in vivo to a self-adjuvanting immunogenic molecule of the invention that comprises a HLA A2-restricted CTL epitope or a vaccine composition comprising same.

Without being bound by any theory or mode of action, the biological effects of the self-adjuvanting immunogenic molecules are exerted through their ability to stimulate and mature dendritic cells. It is the dendritic cells which then activate CD4⁺ and CD8⁺ T cells in the draining lymph nodes.

In a related embodiment, the invention provides a method of enhancing the cell mediated immunity of a subject, said method comprising contacting ex vivo cells, preferably dendritic cells, obtained from a subject with an immunologically active self-adjuvanting immunogenic molecule of the invention or a derivative or variant thereof or a vaccine composition comprising said self-adjuvanting immunogenic molecule or derivative or variant for a time and under conditions sufficient to mature said dendritic cells. Said dendritic cells are then capable of conferring epitope specific activation of T cells and/or B cells.

In a preferred embodiment, the invention provides a method of enhancing the cell mediated immunity of a subject, said method comprising:
(i) contacting ex vivo dendritic cells obtained from a subject with an immunologically active self-adjuvanting immunogenic molecule of the invention or a derivative or variant thereof or a vaccine composition comprising said self-adjuvanting immunogenic molecule or derivative or variant for a time and under conditions sufficient to mature said dendritic cells; and
(ii) introducing the activated dendritic cells autologously to the subject or syngeneically to another subject in order that T cell and/or B cell activation occurs.

The T cell may be a CTL or CTL precursor cell or a CD4⁺ T helper cell.

The subject from whom the dendritic cells are obtained may be the same subject or a different subject to the subject being treated. The subject being treated can be any subject carrying a latent or active infection by a pathogen, such as, for example, a parasite, bacterium or virus or a subject who is otherwise in need of obtaining vaccination against such a pathogen or desirous of obtaining such vaccination. The subject being treated may also be treated for a tumour that they are carrying or may be vaccinated against developing a tumour.

By “epitope specific activity” is meant that the T cell is rendered capable of being activated as defined herein above (i.e. the T cell will recognize and lyze a cell harboring a pathogen from which the CTL epitope is derived, or is able to recognize a T cell epitope of an antigen of a pathogen either transiently or in a sustained manner). Accordingly, it is particularly preferred for the T cell to be a CTL precursor which by the process of the invention is rendered able to recognize and lyze a cell harboring the pathogen or able to recognize a T cell epitope of an antigen of the pathogen either transiently or in a sustained manner.

For such an ex vivo application, the dendritic cells are preferably contained in a biological sample obtained from a subject, such as, for example, blood, PBMC or a buffy coat fraction derived therefrom.

Another aspect of the invention provides a method of providing or enhancing immunity against a pathogen in an uninfected subject comprising administering to said subject an immunologically active self-adjuvanting immunogenic molecule of the invention or a derivative or variant thereof or a vaccine composition comprising said self-adjuvanting immunogenic molecule or derivative or variant for a time and under conditions sufficient to provide immunological memory against a future infection by the pathogen.

In a related embodiment, the invention provides a method of enhancing or conferring immunity against a pathogen in an uninfected subject comprising contacting ex vivo dendritic cells obtained from the subject with an immunologically active self-adjuvanting immunogenic molecule of the invention or a derivative or variant thereof or a vaccine composition comprising said self-adjuvanting immunogenic molecule or derivative or variant for a time and under conditions sufficient to confer epitope specific activity on T cells and/or B cells.

Accordingly, this aspect of the invention provides for the administration of a prophylactic vaccine to the subject, wherein the active substituent of said vaccine (i.e. the self-adjuvanting immunogenic molecule of the invention) induces immunological memory via memory T cells in an uninfected individual. The preferred embodiments of vaccination protocols described herein for enhancing the cell mediated immunity of a subject apply mutatis mutandis to the induction of immunological memory against the pathogen in a subject.

Accordingly, the present invention contemplates providing or enhancing immunity against the following pathogens human immunodeficiency virus (HIV), the human papilloma virus, Epstein-Barr virus, the polio virus, the rabies virus, the Ebola virus, the influenza virus, the encephalitis virus, smallpox virus, the rabies virus, the herpes viruses, the sendai virus, the respitory syncytial virus, the othromyxoviruses, the measles viruses, the vesicular stomatitis virus, visna virus and cytomegalovirus, Acremonium spp., Aspergillus spp., Basidiobolus spp., Bipolaris spp., Blastomyces dermatidis, Candida spp., Cladophialophora carrionii, Coccoidiodes immitis, Conidiobolus spp., Cryptococcus spp., Curvularia spp., Epidermophyton spp., Exophiala jeanselmei, Exserohilum spp., Fonsecaea compacta, Fonsecaea pedrosoi, Fusarium oxysporum, Fusarium solani, Geotrichum candidum, Histoplasma capsulatum var. capsulatum, Histoplasma capsulatum var. duboisii, Hortaea werneckii, Lacazia loboi, Lasiodiplodia theobromae, Leptosphaeria senegalensis, Madurella grisea, Madurella mycetomatis, Malassezia furfur, Microsporum spp., Neotestudina rosatii, Onychocola canadensis, Paracoccidioides brasiliensis, Phialophora verrucosa, Piedraia hortae, Piedra iahortae, Pityriasis versicolor, Pseudallesheria boydii, Pyrenochaeta romeroi, Rhizopus arrhizus, Scopulariopsis brevicaulis, Scytalidium dimidiatum, Sporothrix schenckii, Trichophyton spp., Trichosporon spp., Zygomcete fungi, Absidia corymbifera, Rhizomucor pusillus and Rhizopus arrhizus, Bacillus anthracia, Bordetella pertussis, Vibrio cholerae, Escherichia coli, Shigella dysenteriae, Clostridium perfringens, Clostridium botulinum, Clostridium tetani, Corynebacterium diphtheriae and Pseudomonas aeruginosa.

Another aspect of the invention provides a method of providing or enhancing immunity against a cancer in a subject comprising administering to said subject an immunologically active self-adjuvanting immunogenic molecule of the invention or a derivative or variant thereof or a vaccine composition comprising said self-adjuvanting immunogenic molecule or derivative or variant for a time and under conditions sufficient to provide immunological memory against the cancer.

In a related embodiment, the invention provides a method of enhancing or conferring immunity against a cancer in a subject comprising contacting ex vivo dendritic cells obtained from said subject with an immunologically active self-adjuvanting immunogenic molecule of the invention or a derivative or variant thereof or a vaccine composition comprising said self-adjuvanting immunogenic molecule or derivative or variant for a time and under conditions sufficient to confer epitope specific activity on T cells.

Accordingly, this aspect of the invention provides for the administration of a prophylactic vaccine to the subject, wherein the active substituent of said vaccine (i.e. the self-adjuvanting immunogenic molecule of the invention) induces immunological memory via memory T cells in an individual. The preferred embodiments of vaccination protocols described herein for enhancing the cell mediated immunity of a subject apply mutatis mutandis to the induction of immunological memory against the cancer in a subject.

Accordingly, the present invention contemplates providing or enhancing immunity against the following cancers ABL1 protooncogene, AIDS related cancers, acoustic neuroma, acute lymphocytic leukaemia, acute myeloid leukaemia, adenocystic carcinoma, adrenocortical cancer, agnogenic myeloid metaplasia, alopecia, alveolar soft-part sarcoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumors, breast cancer, CNS tumors, carcinoid tumors, cervical cancer, childhood brain tumors, childhood cancer, childhood leukaemia, childhood soft tissue sarcoma, chondrosarcoma, choriocarcinoma, chronic lymphocytic leukaemia, chronic myeloid leukaemia, colorectal cancers, cutaneous t-cell lymphoma, dermatofibrosarcoma-protuberans, desmoplastic-small-round-cell-tumor, ductal carcinoma, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibrosarcoma, gall bladder cancer, gastric cancer, gastrointestinal cancers, gastrointestinal-carcinoid-tumor, genitourinary cancers, germ cell tumors, gestational-trophoblastic-disease, glioma, gynaecological cancers, haematological malignancies, hairy cell leukaemia, head and neck cancer, hepatocellular cancer, hereditary breast cancer, histiocytosis, Hodgkin's disease, human papillomavirus, hydatidiform mole, hypercalcemia, hypopharynx cancer, intraocular melanoma, islet cell cancer, Kaposi's sarcoma, kidney cancer, Langerhan's-cell-histiocytosis, laryngeal cancer, leiomyosarcoma, leukaemia, li-fraumeni syndrome, lip cancer, liposarcoma, liver cancer, lung cancer, lymphedema, lymphoma, Hodgkin's lymphoma, non-Hodgkin's lymphoma, male breast cancer, malignant-rhabdoid-tumor-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, nasal cancer, nasopharyngeal cancer, nephroblastoma, neuroblastoma, neurofibromatosis, nijmegen breakage syndrome, non-melanoma skin cancer, non-small-cell-lung-cancer-(nscic), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumors, pituitary cancer, polycythemia vera, prostate cancer, rare-cancers-and-associated-disorders, renal cell carcinoma, retinoblastoma, rhabdomyosarcoma, Rothmund-Thomson syndrome, salivary gland cancer, sarcoma, schwannoma, Sezary syndrome, skin cancer, small cell lung cancer (scic), small intestine cancer, soft tissue sarcoma, spinal cord tumors, squamous-cell-carcinoma-(skin), stomach cancer, synovial sarcoma, testicular cancer, thymus cancer, thyroid cancer, transitional-cell-cancer-(bladder), transitional-cell-cancer-(renal-pelvis−/−ureter), trophoblastic cancer, urethral cancer, urinary system cancer, uroplakins, uterine sarcoma, uterus cancer, vaginal cancer, vulva cancer, Waldenstrom's-macroglobulinemia or Wilms' tumor.

According to another embodiment, the pharmaceutical compositions described herein will comprise one or more immunostimulants in addition to the self-adjuvanting immunogenic molecules of this invention. An immunostimulant refers to essentially any substance that enhances or potentiates an immune response (antibody and/or cell-mediated) to an exogenous antigen. One preferred type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Within certain embodiments of the invention, the adjuvant composition induces an immune response predominantly of the Th1 type. High levels of Th1-type cytokines (e.g., IFN-γ, TNFα., IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann et al. Ann Rev Immunol 7:145-173, 1989.

Additional illustrative adjuvants for use in the pharmaceutical compositions of the invention include Montanide ISA 720 (Seppic, France), SAF (Chiron, Calif, United States), ISCOMS (CSL), MF-59 (Chiron), the SBAS series of adjuvants (e.g., SBAS-2 or SBAS-4, available from SmithKline Beecham, Rixensart, Belgium), Detox (Enhanzyn®) (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1.

According to another embodiment of this invention, an immunogenic composition described herein is delivered to a host via antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects or anti-pathogen effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

The present invention uses dendritic cells or progenitors thereof as antigen-presenting cells. Dendritic cells are highly potent APCs (Banchereau et al. Nature 392:245-251, 1998) and have been shown to be effective as a physiological adjuvant for eliciting prophylactic or therapeutic antitumor or anti-pathogen immunity (see Timmerman et al. Ann Rev Med 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate nave T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al. Nature Med 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNF.α., CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ. receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

The development of suitable dosing and treatment regimens for using the particular compositions described herein in a variety of treatment regimens, including e.g., intravenous, intranasal, and intramuscular administration and formulation, is well known in the art, some of which are briefly discussed below for general purposes of illustration.

In certain circumstances it will be desirable to deliver the pharmaceutical compositions disclosed herein parenterally, intravenously, intramuscularly, or even intraperitoneally. Such approaches are well known to the skilled artisan, some of which are further described, for example, in U.S. Pat. No. 5,543,158; U.S. Pat. No. 5,641,515 and U.S. Pat. No. 5,399,363. In certain embodiments, solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. Moreover, for human administration, preparations will of course preferably meet sterility, pyrogenicity, and the general safety and purity standards as required by FDA Office of Biologics standards.

In another embodiment of the invention, the compositions disclosed herein may be formulated in a neutral or salt form. Illustrative pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described, e.g., in U.S. Pat. No. 5,756,353 and U.S. Pat. No. 5,804,212. Likewise, the delivery of drugs using intranasal microparticle resins (Takenaga et al. J Controlled Release 52(1-2): 81-7, 1998) and lysophosphatidyl-glycerol compounds (U.S. Pat. No. 5,725,871) are also well-known in the pharmaceutical arts. Likewise, illustrative transmucosal drug delivery in the form of a polytetrafluoroetheylene support matrix is described in U.S. Pat. No. 5,780,045.

In further aspects of the present invention, the pharmaceutical compositions described herein may be used for the treatment of cancer or a pathogenic infection. Within such methods, the pharmaceutical compositions described herein are administered to a subject, typically a warm-blooded animal, preferably a human. A subject may or may not be afflicted with cancer or a pathogenic infection. Accordingly, the above pharmaceutical compositions may be used to prevent the development of a cancer or to treat a patient afflicted with a cancer or to prevent infection by a pathogen or to treat a pathogenic infection.

Within certain embodiments, immunotherapy may be active immunotherapy, in which treatment relies on the in vivo stimulation of the endogenous host immune system to react against tumors or pathogens with the administration of immune response-modifying agents, such as the self-adjuvanting immunogenic molecules provided herein.

Routes and frequency of administration of the therapeutic compositions described herein, as well as dosage, will vary from individual to individual, and may be readily established using standard techniques. In general, the pharmaceutical compositions and vaccines may be administered by injection (e.g., intracutaneous, intramuscular, intravenous or subcutaneous) or intranasally (e.g., by aspiration). Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor or anti-pathogen immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more polypeptides, the amount of each polypeptide present in a dose ranges from about 25 mg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in pre-existing immune responses to a tumor protein generally correlate with an improved clinical outcome. Such immune responses may generally be evaluated using standard proliferation, cytotoxicity or cytokine assays, which may be performed using samples obtained from a patient before and after treatment.

The self-adjuvanting immunogenic molecules of the invention are readily modified for diagnostic purposes. For example, it is modified by addition of a natural or synthetic hapten, an antibiotic, hormone, steroid, nucleoside, nucleotide, nucleic acid, an enzyme, enzyme substrate, an enzyme inhibitor, biotin, avidin, polyethylene glycol, a peptidic polypeptide moiety (e.g. tuftsin, polylysine), a fluorescence marker (e.g. FITC, RITC, dansyl, luminol or coumarin), a bioluminescence marker, a spin label, an alkaloid, biogenic amine, vitamin, toxin (e.g. digoxin, phalloidin, amanitin, tetrodotoxin), or a complex-forming agent.

The present invention is further described with reference to the following non-limiting examples and drawings. The examples provided herein in mice are accepted models for equivalent diseases in humans and the skilled person will readily be capable of extending the findings presented herein for such models to a human disease context without undue experimentation.

### Example 1

### Materials and Methods

### Chemicals

Unless otherwise stated chemicals were of analytical grade or its equivalent. N,N′-dimethylformamide (DMF), piperidine, trifluoroacetic acid (TFA), O′benzotriazole-N,N,N′,N′-tetramethyluronium hexafluorophosphate (HBTU), 1-hydroxybenzotriazole (HOBt) and diisopropylethylamine (DIPEA) and diisopropylcarbodiimide (DIPCDI) were obtained from Auspep Pty. Ltd., Melbourne, Australia and Sigma-Aldrich Pty. Ltd., Castle Hill, Australia. Dichloromethane (DCM) and diethylether were from Merck Pty Ltd. (Kilsyth, Australia). Phenol and triisopropylsilane (TIPS) were from Aldrich (Milwaulke, Wis.) and trinitrobenzylsulphonic acid (TNBSA) and diaminopyridine (DMAP) from Fluka; 1,8-diazabicyclo[5.4.0]undec-7-ene (DBU) was obtained from Sigma and palmitic acid was from Fluka. The solid support TentaGel S RAM and TentaGel S Am was from Rapp Polymere GmbH, Tubingen, GERMANY. O-(N-Fmoc-2-aminoethyl)-O′-(2-carboxyethyl) undecaethylene glycol (Fmoc-PEG) was obtained from Novabiochem, Merck Biosciences, Switzerland. The heterobifunctional linker molecule N-Succinimidyl 6-maleimidocaproate (MCS) was from Fluka Biochemika, Switzerland. Hen egg lysozyme, ovalbumin and β-galactosidase are from Sigma.

### Synthesis of Water-Soluble Pam2Cys-Based Lipid Moieties Which Can be Used as Modules to Lipidate Protein in Aqueous Solution

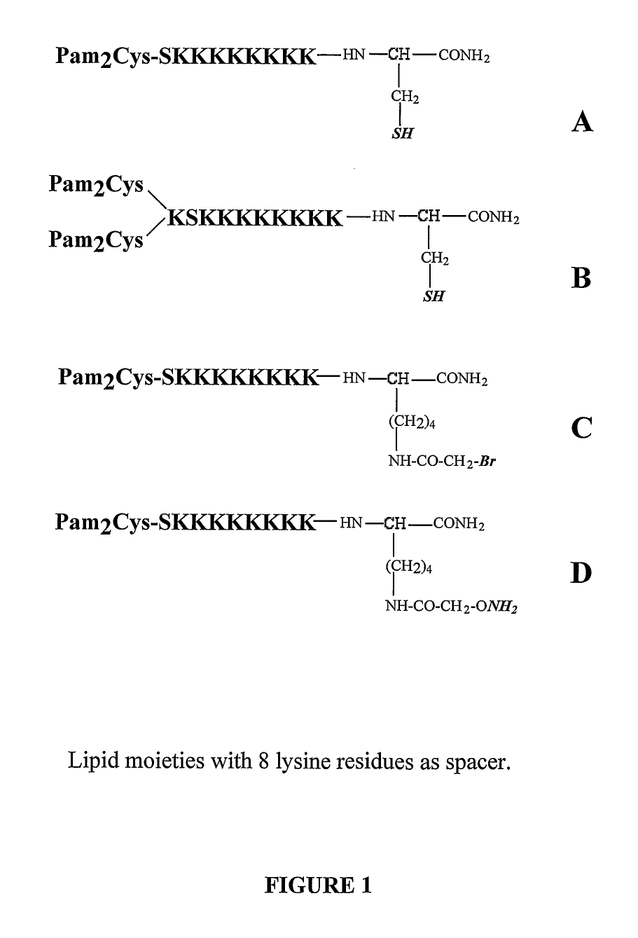

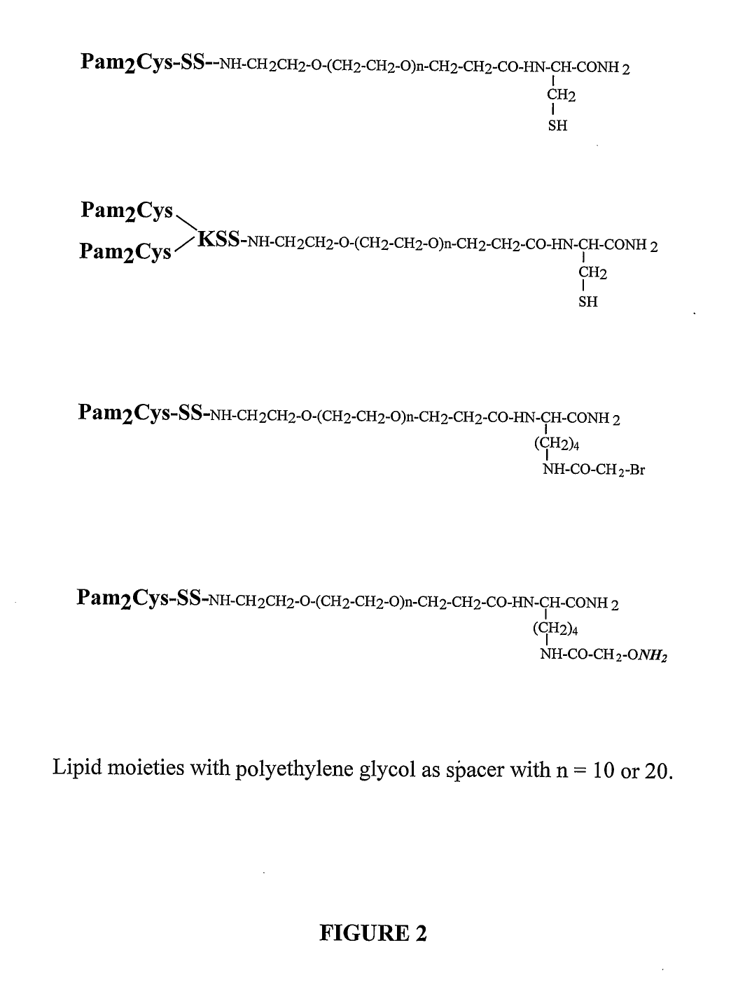

A schematic representation of the water-soluble lipid modules is shown in FIGS. 1 and 2.

The lipid moieties were assembled by conventional solid-phase methodology using Fmoc chemistry. The general procedure used for the peptide synthesis has been described by Jackson et al, Vaccine 18:355, 1999. The solid support TentaGel S RAM was used. Four-fold excess of the Fmoc amino acid derivatives were used in the coupling steps except for the coupling of Fmoc-PEG where only two-fold excess was used.

Pam2Cys was coupled to peptides according to the methods described by Jones et al, Xenobiotica 5:155, 1975 and Metzger et al, Int J Pept Protein Res 38:545, 1991, with the following modifications:

### I. Synthesis of S-(2,3-Dihydroxypropyl)cysteine

Triethylamine (6 g, 8.2 ml, 58 mmoles) was added to L-cysteine hydrochloride (3 g, 19 mmole) and 3-bromo-propan-1,2-diol (4.2 g, 2.36 ml, 27 mmole) in water and the homogeneous solution kept at room temperature for 3 days. The solution was reduced in vacuo at 40° C. to a white residue which was boiled with methanol (100 ml), centrifuged and the residue dissolved in water (5 ml). This aqueous solution was added to acetone (300 ml) and the precipitate isolated by centrifugation. The precipitate was purified by several precipitations from water with acetone to give S-(2,3-dihydroxypropyl)cysteine as a white amorphous powder (2.4 g, 12.3 mmol, 64.7%).

### II. Synthesis of N-Fluorenylmethoxycarbonyl-S-(2,3-dihydroxypropyl)-cysteine (Fmoc-Dhc-OH)

S-(2,3-dihydroxypropyl)cysteine (2.45 g, 12.6 mmole) was dissolved in 9% sodium carbonate (20 ml). A solution of fluorenylmethoxycarbonyl-N-hydroxysuccinimide (3.45 g, 10.5 mmole) in acetonitrile (20 ml) was added and the mixture stirred for 2 h, then diluted with water (240 ml), and extracted with diethyl ether (25 ml×3). The aqueous phase was acidified to pH 2 with concentrated hydrochloric acid and was then extracted with ethyl acetate (70 ml×3). The extract was washed with water (50 ml×2) and saturated sodium chloride solution (50 ml×2), dried over sodium sulfate and evaporated to dryness. Recrystalisation from ether and ethyl acetate at −20° C. yielded a colorless powder (2.8 g, 6.7 mmole, 63.8%).

### III. Coupling of Fmoc-Dhc-OH to Resin-Bound Peptide

Fmoc-Dhc-OH (100 mg, 0.24 mmole) was activated in DCM and DMF (1:1, v/v, 3 ml) with HOBt (36 mg, 0.24 mmole) and DICI (37 ul, 0.24 mmol) at 0° C. for 5 min. The mixture was then added to a vessel containing the resin-bound peptide (0.04 mmole, 0.25 g amino-peptide resin). After shaking for 2 h the solution was removed by filtration and the resin was washed with DCM and DMF (3×30 ml each). The reaction was monitored for completion using the TNBSA test. If necessary a double coupling was performed.

### IV. Palmitoylation of the Two Hydroxy Groups of the Fmoc-Dhc-peptide Resin

Palmitic acid (204 mg, 0.8 mmole), DICI (154 ul, 1 mmole) and DMAP (9.76 mg, 0.08 mmole) were dissolved in 2 ml of DCM and 1 ml of DMF. The resin-bound Fmoc-Dhc-peptide resin (0.04 mmole, 0.25 g) was suspended in this solution and shaken for 16 h at room temperature. The solution was removed by filtration and the resin was then washed with DCM and DMF thoroughly to remove any residue of urea. The removal of the Fmoc group was accomplished with 2.5% DBU (2×5 mins).

All resin-bound peptide constructs were cleaved from the solid phase support with reagent B (88% TFA, 5% phenol, 2% TIPS, 5% water) for 2 hr, and purified by reversed phase chromatography as described by Zeng et al., Vaccine 18, 1031 (2000).

Analytical reversed phase high pressure liquid chromatography (RP-HPLC) was carried out using a Vydac C4 column (4.6×300 mm) installed in a Waters HPLC system and developed at a flow rate of 1 ml/min using 0.1% TFA in H2O and 0.1% TFA in CH3CN as the limit solvent. All products presented as a single major peak on analytical RP-HPLC and had the expected mass when analysed by Agilent 1100 LC-MSD trap mass spectrometer.

### Synthesis of Construct A and B (FIG. 1)

The resin TentaGel S Am resin was used. Fmoc-Cys(Trt)-OH was used as the first amino acid to be coupled to the resin and then followed with 8 Fmoc-Lys(Boc)-OH and Fmoc-Ser(tBu)-OH. For synthesis of Construct A Fmoc-S-(2,3-bis-hydroxy-2-propyl)-cysteine [Fmoc-Cys(Dhc)-OH] was coupled to the serine residue before the palmitoylation with palmitic acid in the presence of dimethylaminopyridine (DMAP) and diisopropylcarbodiimide for 16 hrs. For the synthesis of Construct B Fmoc-Lys(Fmoc)-OH was coupled to the serine residue. Following the removal of both the Fmoc groups Fmoc-Cys(Dhc)-OH was coupled to the two exposed amino groups before the palmitoylation with palmitic acid in the presence of dimethylaminopyridine (DMAP) and diisopropylcarbodiimide for 16 hrs. At the end of the synthesis the Fmoc group of the cysteine residue was removed peptides were cleaved from the resins and side chain deprotected to generate Construct A and B.

### Synthesis of Construct C and D (FIG. 1)

The resin TentaGel S Am resin was used. Fmoc-Lys(Mtt)-OH was used as the first amino acid to be coupled to the resin and then followed with 8 Fmoc-Lys(Boc)-OH and Fmoc-Ser(tBu)-OH. Fmoc-Cys(Dhc)-OH was coupled to the serine residue before the palmitoylation with palmitic acid in the presence of dimethylaminopyridine (DMAP) and diisopropylcarbodiimide for 16 hrs. Following removal of the Fmoc group the N-terminal amino group was blocked using Di-t-butyl di-carbonate. The Mtt group was selectively removed using 1% trifluoroacetic acid in dichloromethane. For synthesis of Construct C bromoacetic acid was coupled to the exposed amino group under the activation of disiopropylcarbodiimide (DIC). For synthesis of Construct D Boc-aminooxyacetic acid was coupled to the exposed amino group. Peptides were cleaved from the resins and side chain and deprotected to generate Construct C and D.

These four constructs have 8 lysines to help to increase the solubility of the lipid moieties.
Construct A has one copy of Pam2Cys per lipid module.
Construct B has two copies of Pam2Cys per lipid module. This module could be useful in those cases where the sites of available for lipidation are limited.
Construct C can be used to couple directly to any free SH groups within proteins or recombinant proteins.
Construct D has an aminooxy group which forms an oxime bond with an aldehyde function group which can be generated by oxidizing a serine residue existing or being engineered in at the N-terminal of a protein or recombinant protein.

Four lipid moiety analogues with polyethylene glycol as spacer (FIG. 2) were synthesised following the protocol described above and they can be used to lipidate protein in a similar way as described above for Construct A, B, C and D.

### Example 2

### Synthesis of Four Different Species of Lipidated HEL (Lysozyme) Proteins

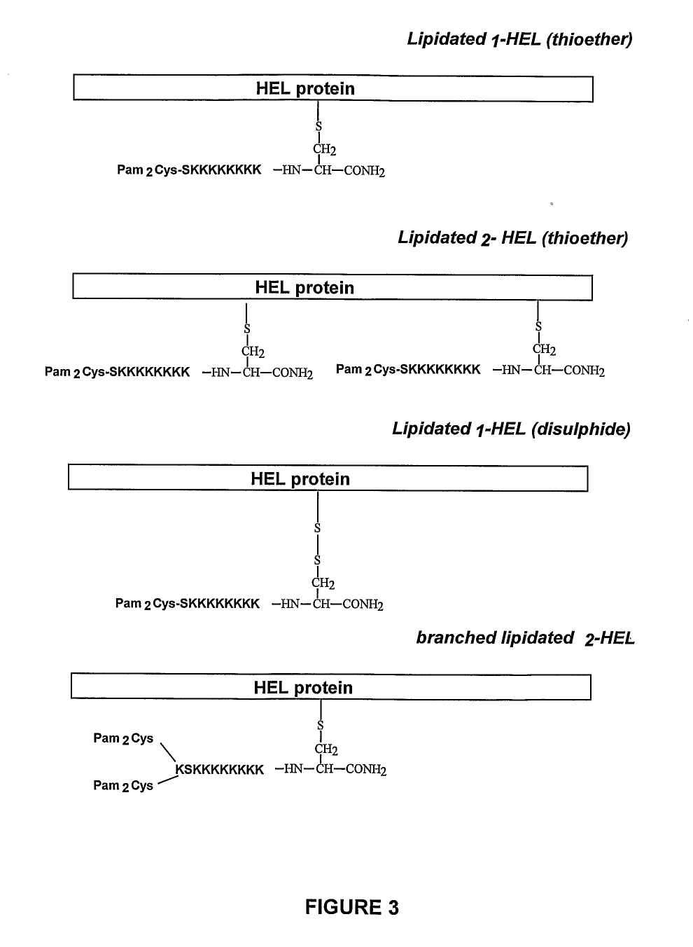

Four different lipidated HEL were prepared by coupling the four lipid moieties listed in FIG. 1 to hen eggwhite lysozyme (HEL) protein. FIG. 3 shows the schematic diagram of these four lipidated HELs

The lipidated HEL proteins Lipidated₁-HEL (thioether) and Lipidated₂-HEL (thioether) were prepared by derivitising HEL with MCS and then chemoselectively ligating the sulfhydryl group of construct A to form a thioether bond between the protein and lipid module. They are different in that Lipidated₂-HEL (thioether) has two copies of construct A. The Branched lipidated₂-HEL has a single copy of the lipid module per protein molecule but there are two copies of pam2cys per protein due to the bivalent nature of construct B. This is more hydrophobic and eluted much later on HPLC.

In order to make Lipidated₁-HEL (disulphide) HEL was modified with the heterobifunctional linker 3-(2-pyridyldithio)propionic acid N-hydroxysuccinimide ester (SPDP) and then reacted with construct A to generate Lipidatedᵢ-HEL (disulphide) by formation of a disulphiide bond between the lipid module and protein.

### Example 3

### Evaluation of Immunogenicity of Lipidated Insulin

Insulin was lipidated using the following procedures. 10 mg of bovine pancreatic insulin was dissolved in 4000 of 6M guanidine hydrochloride containing 0.5M phosphate buffer (pH 7.9) and 4000 of 0.02M phosphate buffer (pH 7). To this solution was added 3.25 mg of N-Succinimidyl 6-maleimidocaproate (MCS) in 2000 of acetonitrile and after 3 hrs the MCS-modified insulin was isolated by semi-preparative HPLC. 3.3 mg of the MCS-modified insulin and 5 mg of Pam2CysSer(Lys)8Cys were dissolved in 5000 of acetonitrile and 500 μl of water. The reaction mixture was left at room temperature for 48 h. Two lipidated insulin compounds were isolated by semi-preparative HPLC. Analysis of these species by mass spectrometry demonstrated that\two different lipidated insulins were made, which differed in the number of lipid moieties incorporated per molecule of protein. Pam2Cys₂-insulin had two copies of Pam₂CysSer(Lys)₈Cys per insulin and Pam2Cys₃-insulin with three copies of Pam₂CysSer(Lys)₈Cys per insulin.

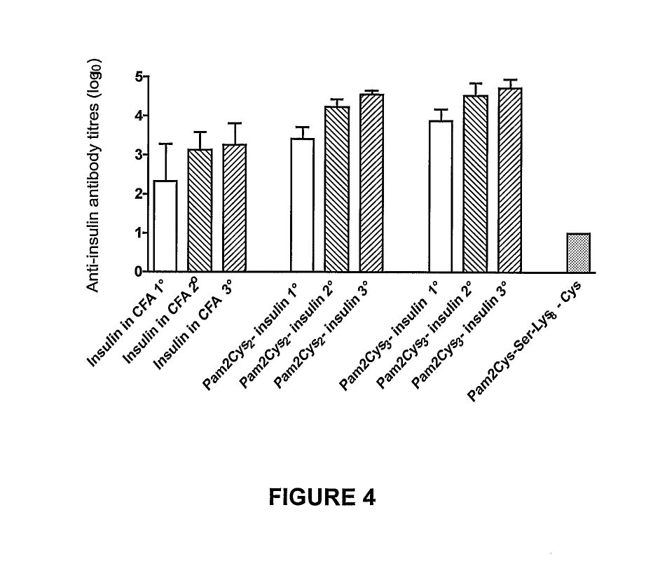

Animal study was performed where mice were inoculated with the lipidated insulins and the antibody response measured. Briefly, four groups of Balb/c mice were inoculated with insulin in Freund's adjuvant (complete for the first dose and incomplete for the second dose, CFA/IFA), Pam2Cys₂-insulin in PBS, Pam2Cys₃-insulin in PBS or the lipid moiety itself in PBS at weeks 0 and 4 and sera was prepared from blood taken at weeks 4, 5 and 6. Serum anti-insulin antibody titers were determined using ELISA. The results demonstrate that the lipidated insulin proteins induced strong anti-insulin responses after a single inoculation which was as strong as those elicited by insulin in CFA/IFA after two inoculations. Following two inoculations with the lipidated insulin, antibody levels were also significantly higher than those seen in the CFA/IFA group. The lipidated insulin with 3 copies of the lipid moiety was found to be more immunogenic that the insulin with two copies. The results are shown in FIG. 4.

### Example 4

### Antibody Induction by the Lipidated Protein Hen Egg Lysozyme (HEL)

### Step 1: Modification of HEL with N-Succinimidyl 6-maleimidocaproate

15 mg of HEL (Mr=14305) was dissolved in 800 ul of 6 M Guanidine buffer (pH=7.75) and to this solution was added 1.30 mg of N-Succinimidyl 6-maleimidocaproate in 200 ul of acetonitrile. The reaction mixture was left at room temperature for 30 mins and the product was isolated by HPLC.

### Step 2: Conjugation of Pam2Cys moiety to the MCS-modified HEL

3.4 mg of the MCS-modified HEL and 1.76 mg of Pam2Cys-SerLys8Cys (SEQ ID NO:7) were dissolved in 500 ul of 8 M urea in 0.05 M phosphate buffer pH 7.30. The reaction mixture was left at room temperature for 18 hrs and the lipidated HEL containing one copy of Pam2Cys was isolated by HPLC.

### Step 3: Antibody Induction by the Lipidated Protein Hen Egg Lysozyme (HEL)

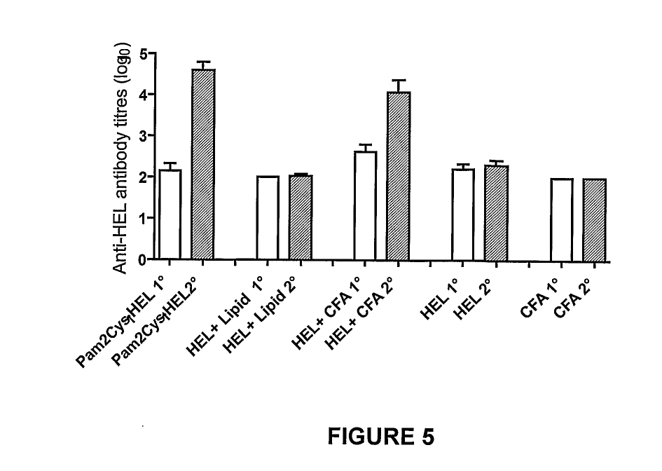

C57BL6 mice were inoculated with the lipidated HEL Pam2Cys1-HEL, HEL and Pam2CysSerLyy8Cys co-admixed, HEL in CFA, HEL in saline and CFA respectively. The mice were given two doses of 30 ug of the immunogens at days 0 and 21 and sera were prepared from the bloods obtained on day 21 and 34. The anti-HEL antibody titres in the sera on day 21 (1°) and 34 (2°) were determined by ELISA (FIG. 5). The results show that the lipidated HEL induced a strong anti-HEL antibody response which is as strong as those obtained when HEL was administered in Freund's adjuvant. In contrast no specific antibody response was elicited when HEL was co-admixed with the lipid moiety.

### Example 5

### Lipidated HEL Induces Anti-HEL Antibody Responses in Two Different Strains of Mice

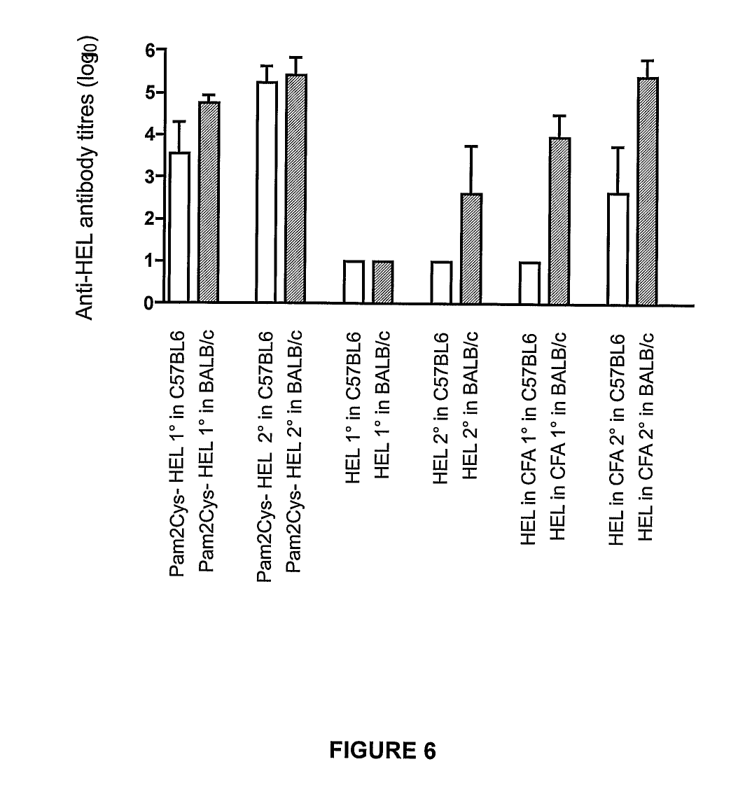

C57BL/6 and BALB/c mice were given two doses of 25 μg of lipidated HEL at week 0 and week 3. As comparison HEL in Freund's adjuvant (the complete for the first inoculation and incomplete for the second inoculation) or HEL in saline alone were given to mice in a same inoculation regime. These mice were bled at week 3 and week 5 and sera were prepared from the bleeds, and anti-HEL antibody responses were determined using ELISAs. The results (FIG. 6) show that lipidated HEL induced strong anti-HEL antibody responses in both strains of mice. The responses were as strong as, if not better than, those obtained when HEL was administered in Freund's adjuvant.

Antibody Responses Induced by Lipidated HEL in which Pam2Cys is Attached to the Protein by Different Chemical Linkers.

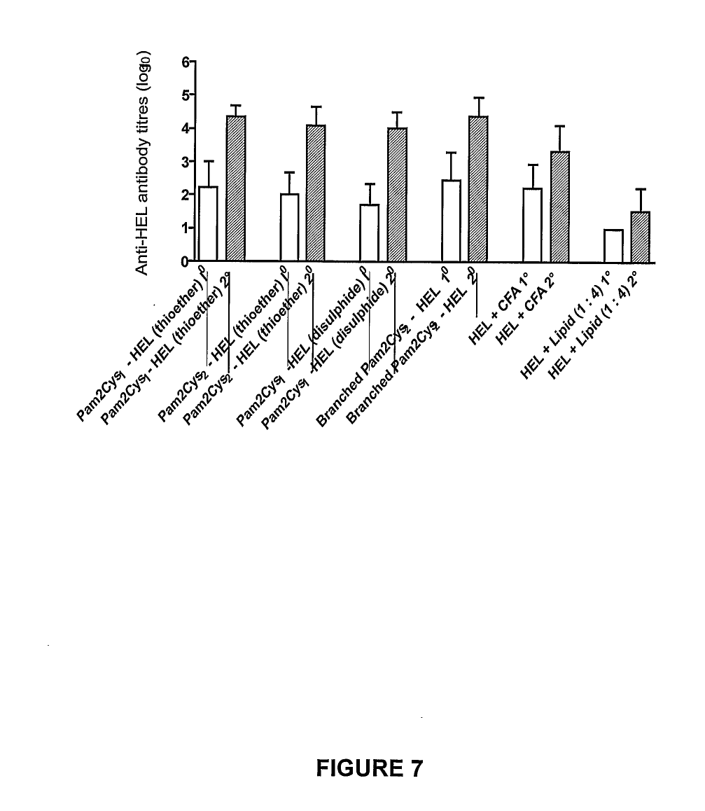

Four different lipidated HELs (FIG. 3) and obtained using different chemical linkers were used to inoculate C57BL/6 mice. Mice received two doses (25 μg each) at weeks 0 and 3. Blood samples were obtained at weeks 0, 3 and 5. Sera were prepared and anti-HEL antibody responses determined by ELISA. One group of mice received two doses of HEL emulsified in complete Freund's adjuvant for the first inoculation and in incomplete Freund's adjuvant for the second inoculation. The results (FIG. 7) show that similar specific anti-HEL antibody responses were obtained irrespective of the chemical linkage used.

### Antibody Induction is T Dependent

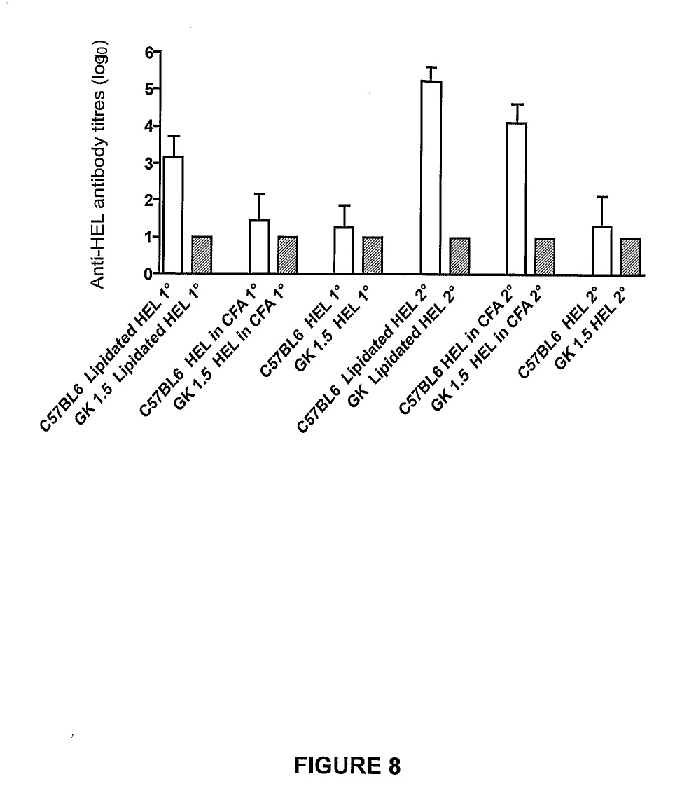

An examination of the antibody isotype profile induced by lipidated HEL (FIG. 8) indicates that the immune response is T cell-dependent. To gain further evidence of the involvement of T helper cells GK 1.5 transgenic mice which lack CD4⁺ T cells were inoculated with the lipidated HEL. As a comparison, wild type C57BL/6 mice were inoculated in parallel with antigen. Mice received two doses (25 μg each dose) at weeks 0 and 4 and were bled at weeks 4 and 6. Anti-HEL antibody titres were determined by ELISA in the sera obtained from the bleeds. The results (FIG. 8) indicate that lipidated HEL induces little or no anti-HEL antibody responses in GK1.5 mice. In contrast a strong anti-HEL antibody response was detected in C57BL/6 mice inoculated with lipidated HEL. GK 1.5 mice receiving two doses of HEL in Freund's adjuvant had little or no anti-HEL antibody (FIG. 8).

### Comparison of the Antibody Responses to HEL in Presence of Lipidation Alum and Freund's Adjuvant.

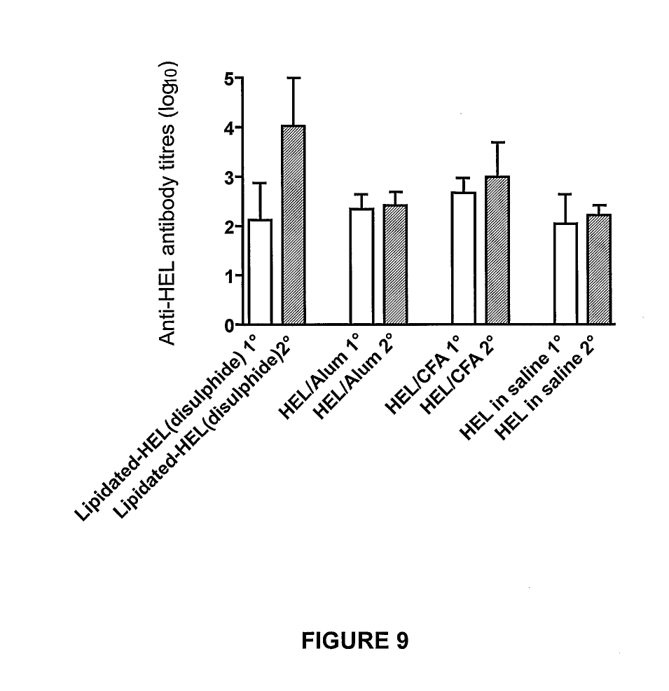

Lipidated HELP, HEL/ALUM and HEL/CFA and HEL/Saline were compared for their ability to induce an antibody response. The results are shown in FIG. 9. Lipidated HEL induced a greater antibody response compared to HEL in Alum, CFA or saline.

### Comparison of the Antibody Isotypes Induced by Lipidated HEL and HEL Administered in Freund's Adjuvant.

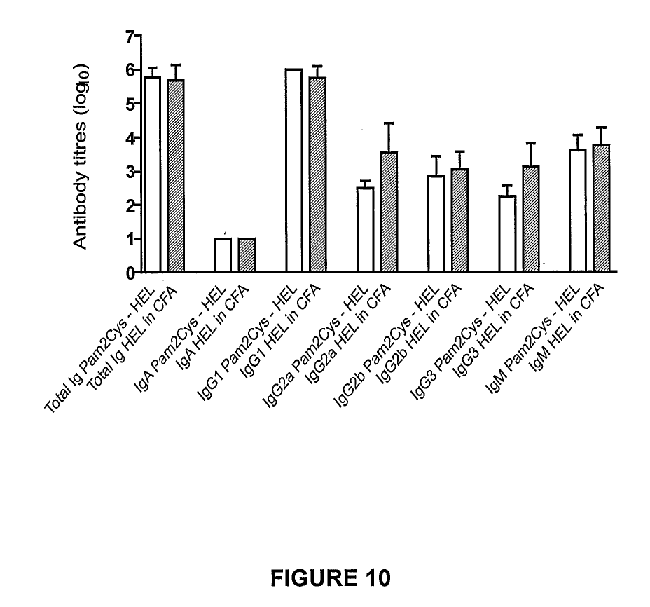

BALB/c mice were inoculated sub-cutaneously with two doses (30 ug each dose) Pam2Cys in saline or HEL emulsified in Freund's adjuvant (complete for the first dose and incomplete for the second dose) on days 0 and 28. Animals were bled 14 days following the second dose of antigen, sera prepared and the isotype of anti-HEL antibodies were determined by ELISA (FIG. 10). The results show that a similar profile of the isotypes were obtained.

### Example 6

### Lipidation of Ovalbumin

6.4 mg of ovalbumin was dissolved in 8M urea in 0.05M phosphate buffer (pH 8.3). To this solution was added 5 mg of dithiodithreitol. The solution was held at 37° C. overnight. The reduced ovalbumin was isolated by gel filtration chromatography on a Superdex G75 10/300GL column using 50 mM ammonium bicarbonate as the eluting buffer (flow rate 0.5 ml/min). The material eluting with a retention time of 25 mins was collected and concentrated to 1 ml using a spin column (Viva Spin 20 [VIVASCIENCE], molecular weight cut-off 10,000 Da or Ultra-15 [Millipore], molecular weight cut-off 10,000 Da).

The amount of free SH group was determined as follows: to 50 μl of the solution of, reduced protein solution was added 50 μl of 10 mM 5,5′-dithio-bis-(2-nitrobenzoic acid) in 0.1 M phosphate buffer (pH 8). The solution was held at 37° C. for 10 mins and then 900 μl of 50 mM of ammonium hydrogen carbonate was added. The optical density was measured at 412 nm using 50 μl of 10 mM 5,5′-dithio-bis-(2-nitrobenzoic acid) in 0.1 M phosphate buffer (pH 8) added to 950 μl of 5 mM of ammonium hydrogen carbonate as a blank. The amount of free SH group was calculated by the following formula:

optical density/13.6×20×100

50 mg of deoxycholate was added to and dissolved in the reduced protein solution. 1.3 mg of bromoacetylated Pam2CysSK8K (Construct C in FIG. 1) in 200 μl of water was then slowly added to the protein solution. 1 to 3 μl of 10M sodium hydroxide was added to adjust the pH to approximately 8.5. The reaction mixture was held at 37° C. overnight. The final product was isolated using gel permeation chromatography on a column of Superdex G-75 10/300GL using 0.15% w/v deoxycholate in 50 mM ammonium acetate as the elution buffer (flow rate 0.5 ml/min). Fractions were collected and concentrated to 1 ml using VivaSpin 20. The amount of lipidated ovalbumin was determined by UV spectrometry against series of ovalbumin solutions made as standards.

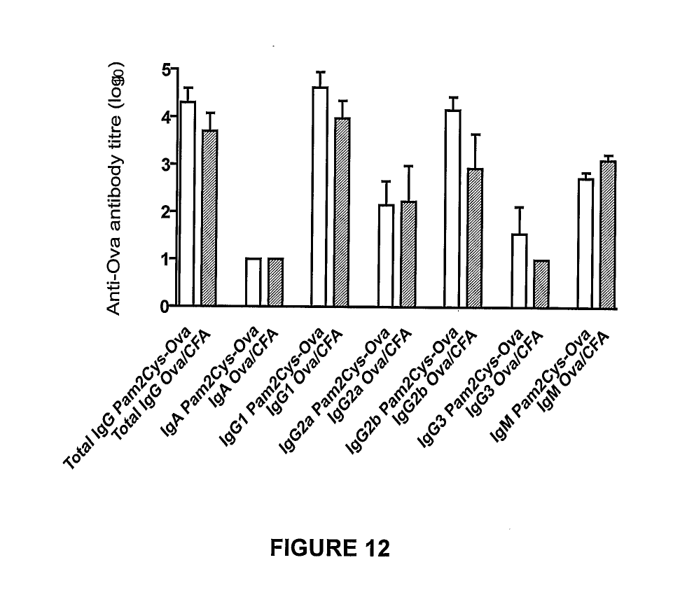

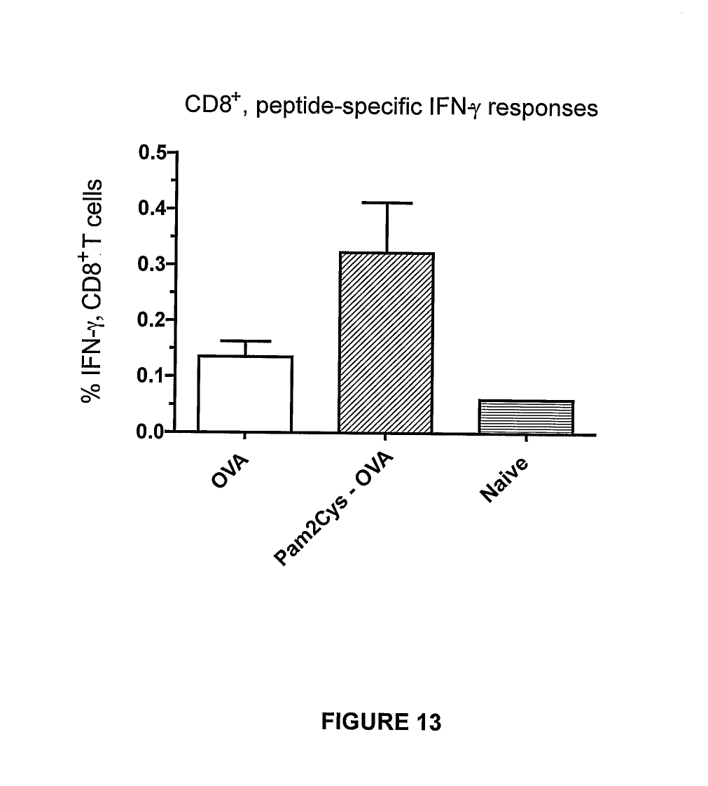

The immunogenic properties of the lipidated ovalbumin were determined by inoculating mice with this material and determining antibody titres (FIGS. 10 and 12) and cytotoxic T cell activity (FIG. 13).

### Example 7

### Lipidation of β-galactosidase

4.86 mg of β-galactosidase was dissolved in 900 μl of 0.1M phosphate buffer (pH 8.0) and to this solution was added 0.70 mg of N-succinimidyl 6-maleimidocaproate (MCS) in 70 μl of acetonitrile. The reaction mixture was held at room temperature for 4 hr. The MCS-modified (3-galactosidase was isolated using Superdex G-75 10/300GL with 50 mM of ammonium acetate as the elution buffer at a flow rate of 0.5 ml/min. Fractions were collected, pooled and concentrated to 1 ml using Viva Spin 20 (molecular weight cut off 10,000 Da.).

To determine the amount of the maleimido groups attached to the β-galactosidase protein, 100 of 5 mM 2-mercaptoethanol was added to 500 of MCS-modified β-galactosidase solution and the mixture held at 37° C. for 7-10 mins. 500 of 10 mM 5,5′-dithio-bis-(2-nitrobenzoic acid) in 0.1 M phosphate buffer (pH 8) was then added followed by 8900 of 0.1 M phosphate buffer (pH 8). The optical density (A) at 412 nm was determined. 10 μl of 5 mM 2-mercaptoethanol was added to 50 μl of 10 mM of 5,5′-dithio-bis-(2-nitrobenzoic acid) in 0.1 M phosphate buffer (pH 8) and after 5 mins at room temperature 940 μl of 0.1 M phosphate buffer (pH 8) was added and the optical density (B) at 412 nm determined. The amount of maleimido groups attached to the β-galactosidase was calculated using the formula:

nmoles maleimide/β-galactosidase=(A−B)/13.6×20×1000

75 mg of deoxycholate was dissolved into 1 ml MCS-modified β-galactosidase and 1.1 mg Pam2CysSK8C in 200 μl of water was slowly added. 1-3 μl of 10M sodium hydroxide was added to adjust the pH to approximately 8.5. The reaction mixture was held at 37° C. overnight. The final product was isolated using Superdex G-75 10/300GL with 0.15% deoxycholate in 50 mM ammonium acetate as the elution buffer at a flow rate of 0.5 ml/min. Fractions were collected and concentrated to 1 ml using Viva Spin 20 (molecular weight cut off 10,000 Da.). The amount of β-galactosidase was determined by UV spectrometry using a series of β-galactosidase solutions as reference.

The efficacy of the vaccine system described here relies on the targeting properties that Pam2Cys has for Toll like receptor 2. This receptor is present on dendritic cells which are particularly efficient at taking up and processing antigen.

### Example 8

### CTL Induction by Lipidated Polytope Using IFN-γ-ELISpot Assays

The polytope has six different CTL epitopes with the sequence

| | |
|---|---|
| YPHFMPTNL, | (SEQ ID NO: 1) |
| SGPSNTPPEI, | (SEQ ID NO: 2) |
| FAPGNYPAL, | (SEQ ID NO: 3) |
| SYIPSAEKI, | (SEQ ID NO: 4) |
| EEGAIVGEI | (SEQ ID NO: 5) |
| and | |
| RPQASGVYM. | (SEQ ID NO: 6) |

1). Lipidation of polytope:
a) Modification of polytope with N-Succinimidyl 6-maleimidocaproate:
Polytope stock solution: 2.13 mg/ml in PBS;
N-Succinimidyl 6-maleimidocaproate (MCS) stock solution: 0.92 mg/ml in acetonitrile.

To 100 μul of polytope stock solution was added 48 μul of MCS stock solution (5 fold excess). The reaction was left at room temperature for 2 hrs. The modified polytope was isolated using HPLC.

b) Conjugation of Pam2Cys moiety to the MCS-modified polytope; The MCS-modified polytope was dissolved in acetonitrile and PBS, and to this solution two-fold excess of Pam2Cys-Ser-(Lys)8-Cys was added. The reaction was left at room temperature for 18 hrs. The lipidated polytope was isolated by HPLC.
2) IFN-γ-ELISpot assays

Epitope tested=SYIPSAEKI(SEQ ID NO:4)(P. berghi circumsporazoite protein):

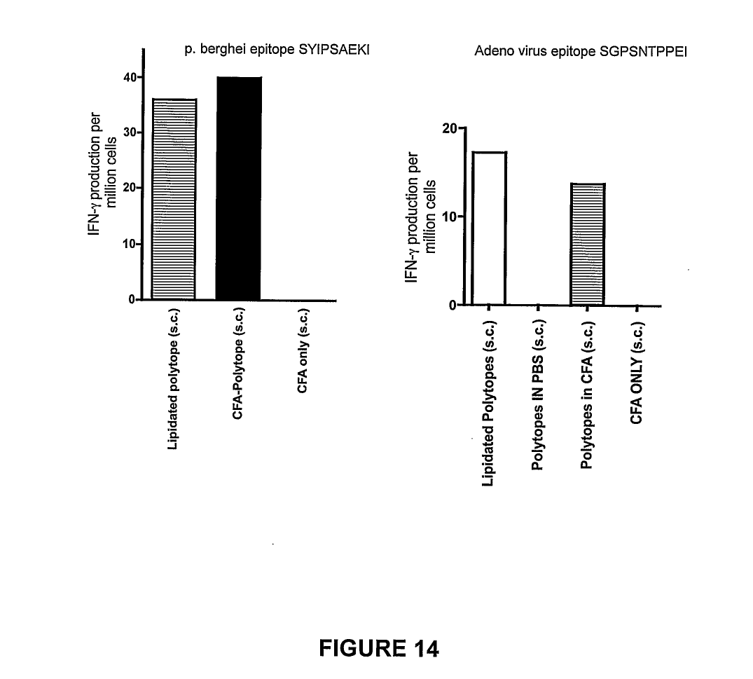

BALB/c mice were inoculated at a dose of 5 nmole/mouse at the base of the tail. Seven days later the spleen was taken and single cell suspension was made (effector cells). IFN-γ-ELISPOT assay performed using a ranging of concentrations of effector. The effector cells were cultured with irradiated autologous spleen cells in the presence or absence of the CTL determinant from P. berghei circumsporazoite protein (249-257) and IFN-γ-ELISpot assays were carried out. The result are shown in FIG. 14.

Epitope tested=SGPSNTPPEI(SEQ ID NO:2)(h-2Db-Adenovirus 5EIA)

C57BL6 mice were inoculated at a dose of 5 nmole/mouse at the base of the tail. 7 days later the spleen was taken and single cell suspension was made (effector cells). IFN-γ-ELISPOT assay performed using a ranging of concentrations of effector. The effector cells were cultured with irradiated autologous spleen cells in the presence or absence of the CTL determinant SGPSNTPPEI (SEQ ID NO: 2) and IFN-γ-ELISpot assays were carried out (FIG. 14).

### Example 9

### Expression of a Recombinant Protein Carrying a Serine Residue at the N-Terminal Position

To conjugate the Pam2Cys molecule to a recombinant protein there is the need for the mature protein molecule to begin with a serine residue as opposed to the normal Methionine residue (arising from the start codon). To do this, the mature protein is expressed and purified in a way such that the protein is transcribed/translated in the normal manner using a methionine residue as the start codon—the protein is then digested with a specific protease to leave a serine residue as the amino-terminal residue.

A protease is selected that is capable of cleaving a protein such that a serine residue is naturally left as the amino-terminal amino acid, or digests proteins that are engineered to incorporate a serine residue at the amino-terminus of the protein after proteolysis. Proteases that fit this criteria include enterokinase and the Factor Xa protease. Both of these proteases have a cleavage site that does not require a specific amino acid to follow the point of cleavage but may not cut if certain residues are present.

An expression vector is then chosen which allows for the cloning, expression, purification and cleavage of the recombinant protein of choice. The pET30(a/b/c) series of vectors fits this criteria. It is an expression vector that is inducible by IPTG, incorporates a multiple cloning site that allows the cloning of a gene such that the protein expressed will have either an N- or C-terminal His tag that can be used for purification and an enterokinase site is present that allows cleavage of the mature protein once it is purified.

The sequence surrounding the enterokinase cleavage site and multiple cloning site must be manipulated. This manipulation allows DNA encoding the protein of choice to be ligated in-frame behind the enterokinase cleavage site that has a serine residue incorporated directly downstream. This allows for expression of the protein of choice by utilising the promoter region of the pET30 vector, an N-terminal His tag is then present which allows for purification and the purified protein can then be cleaved using enterokinase. Once cleaved, the His tag is removed and the mature protein will have a serine residue as the first amino acid.

Two proteins were chosen to test the pET30 construct that was generated. These proteins were ovalbumin from hen egg lysozyme and gB from Herpes Simplex Virus. Oligonucleotides were designed to PCR amplify the genes so that all transmembrane domains and signal peptides were removed, this was done in an attempt to obtain a more soluble form of the protein once purified. The expression of the proteins is induced using IPTG and then the His tag is utilised to purify the protein on a Nickel resin. Following purification the protein is cleaved with enterokinase and the protease is then removed using an enterokinase capture resin. The resulting protein is in a soluble form with a serine residue as the primary amino acid. This protein is then subjected to the lipid ligation chemistry described below.

### Example 10

### Cloning and Expression of Glycoprotein B (gB) from Herpes Simplex Virus

The gB protein with a N-terminal serine was expressed using the method described as Example 9.

The lipidation of gB: the gB expressed from E. coli carrying a serine reside at its N-terminal position is oxidised using sodium periodate to generate an aldehyde function group on its N-terminus. This oxidised gB reacts with the lipid moiety D to form an oxime bond.

### Immunisation and Viral Infection

C57BL6 mice is immunized with the lipidated gB dissolved in saline intranasally. For the viral challenge the mice is infected with HSV-KOS using flank scarification or by intranasal inoculation. Viral titres were determined using standard PFU assays on confluenct Vero cell monolayers. Samples were taken from the lungs (i.n. inoculation) or viral infection site (flank scarification) and homogenised, and 10-fold serial dilutions are then tested for plaque formation to determine viral titre in the original tissue.

The epitope-specific CD8+ positive T cells are assessed by tetramer staining. H-2 Kb-gB498-5-5 tetramers are prepared as described in Jones et al. J Virol 74:2414-9, 2000.

### Example 11

### Cloning and Expression of Ovalbumin

The ovalbumin with a serine residue at its N-terminus was expressed using the method described in Example 9.

### Lipidation of Ovabumin

The ovalbumin expressed from E. coli carrying a serine reside at its N-terminal position is oxidised using sodium periodate to generate an aldehyde function group on its N-terminus. This oxidised ovalbumin reacts with Construct D (FIG. 1) to form an oxime bond. Alternatively ovalbumin protein can be lipidated using other methods described in the examples.

### CTL Experiment

C57BL6 mice are inoculated with lipidated ovalbumin subcutaneously. Interferon-γγ ELIspot assays are carried out with single cell suspension prepared from the organs such as spleen or lymph nodes.

### Example 12

### Immune Responses Induced by Lipidated β-galactosidase

C57BL6 mice are injected with lipidated β-galactosidase, administered sub-cutaneously in the scruff of neck, on days 0 and 7. On day 14 the mice are killed, spleens removed and a single cell suspension prepared. The splenic cells are stimulated in vitro with the β-galactosidase peptide epitope TPHPARIGL and the cytotoxic lymphocyte response determined using a peptide-specific IFN-γ assay.

It is expected that the splenocyte preparation will exhibit IFN-γ production as a result of the vaccination regime as exemplified by Example 3.

BALB/c mice receiving two doses of lipidated β-galactosidase on days 0 and 7, also administered sub-cutaneously in the scruff of neck are also expected to demonstrate a cytotoxic T cell response when splenocytes are stimulated in vitro with the peptide epitope DAPIYTNVT.

These results demonstrate that a lipidated protein antigen can induce cytotoxic T cell responses restricted by different major histocompatibility alleles. Antibody responses are also expected to be obtained in both animal strains similar to the results reported for HEL in FIG. 2.

### Example 13

### Lipidation of HBsAg

Hepatitis B small antigen (HBsAg) can be lipidated in a similar way to that described for insulin, HEL or OVA (Examples 2, 3 and 4).

### The CTL Response Induced by Lipidated HBsAg

It is expected that BALB/c mice when inoculated with lipidated HBsAg will demonstrate cytotoxic T cell responses. Splenocytes obtained from inoculated animals will respond to the peptide epitope IPQSLDSWWTSL. It is also expected that mice of different MHC specificities will also respond to their respective class I-restricted peptide epitopes.

### The Antibody Response Induced by Lipidated HBsAg

Animals of different species and strains are also expected to induce antibody in response to inoculation with lipidated HBsAg.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Altman et al. Science 274, 94-96, 1996 Ausubel et al. Current Protocols in Molecular Biology, John Wiley & Sons, New York. N.Y., 1989
Banchereau et al. Nature 392:245-251, 1998
Bodinier et al. Nature Med 6:707-710, 2000
Broglie et al. Science 224:838-843, 1984
Chen et al. Cancer Res 54:1065-1070, 1994
Coligan et al. Current Protocols in Immunology, vol. 1, Wiley Interscience, Greene, 1998
Coruzzi et al. EMBO J. 3:1671-1680, 1984
Creighton Proteins: Structure and Molecular Properties, W. H. Freeman & Co., San Francisco, pp. 79-86, 1983
Dawson et al. Science 266:243-247, 1994
Deres et al. Nature 342:561, 1989
Engelhard et al. Proc Natl Acad Sci 91:3224-3227, 1994
Grant et al. Methods Enzymol 153:516-544, 1987
Harlow and Lane, Antibodies: A Laboratory Manual, Cold Spring Harbor Laboratory, 1988
Hobbs or Murry McGraw Hill Yearbook of Science and Technology, McGraw Hill, New York, N.Y.; pp. 191-196, 1992
Jackson et al, Vaccine 18:355, 1999
Jones et al. J Virol 74:2414-9, 2000
Jones et al. Xenobiotica 5:155, 1975
Kroll et al. DNA Cell Biol 12:441-453, 1993
Logan et al. Proc Natl Acad Sci 81:3655-3659, 1984
Metzger et al. Int J Pept Protein Res. 38: 545, 1991
Metzger et al. J Pept Sci 1:184, 1995
Mosmann et al. Ann Rev Immunol 7:145-173, 1989
Muhlradt et al. J Exp Med 185:1951, 1997
Muhlradt et al. Infect Immun 66:4804, 1998)
Muir et al. Proc Nall Acad Sci USA 95:6705-6710, 1998
Nardin et al J Immunol. 166: 481, 2001
Nardin et al. Vaccine 16: 590, 1998
Ogg et al. Curr Opin Immunol 10: 393-396, 1998
Paul Fundamental Immunology, 3rd ed., 243-247 (Raven Press) 1993
Petrovsky et al. J Immunol. Methods 186:37-46, 1995
Porath et al. Prot Exp Purif 3:263-281, 1992
Powell and Newman, eds., Vaccine Design (the subunit and adjuvant approach), Plenum Press, NY, 1995
Remington's Pharmaceutical Sciences 15th Edition, pages 1035-1038 and 1570-1580
Rose et al. Bioconjug Chem 7:552, 1996
Rose et al. Mol Immunol 32:1031, 1995
Sacht et al. Eur J Immunol 28:4207, 1998
Sambrook et al. Molecular Cloning, A Laboratory Manual, Cold Spring Harbor Press, Plainview, N.Y., 1989
Scharf et al. Results Probl Cell Differ 20:125-162, 1994
Takamatsu EMBO J. 6:307-311, 1987
Takenaga et al. J Controlled Release 52(1-2):81-7, Mar. 2 1998
Tam et al. Biopolymers (Peptide Science) 51:311-332, 1999
Timmerman et al. Ann Rev Med 50:507-529, 1999
Van Heeke et al. J Biol Chem 264:5503-5509, 1989
Vitiello et al. J Exp Med 173:1007, 1991
Wiesmuller et al. Hoppe Seylers Zur Physiol Chem 364:593, 1983
Wiesmuller et al. Vaccine 7:29, 1989
Winter et al. Results Probl Cell Differ 17:85-105, 1991
Zeng et al. J Pept Sci 2:66, 1996
Zeng et al. Vaccine 18:1031, 2000
Zitvogel et al. Nature Med 4:594-600, 1998

1. A method for generating a vaccine said method comprising selecting or preparing a nucleic acid-derived polypeptide and conjugating at least one lipid or fatty acid moiety to any amino acid residue within the polypeptide or to a post translationally added chemical moiety on the nucleic acid-derived polypeptide to create a self-adjuvanting immunogenic polypeptide which induces an immune response across histocompatibility types.

2. The method of claim 1 wherein the chemical moiety is a carbohydrate entity.

3. The method of claim 1 wherein the lipid or fatty acid moiety is conjugated to a side-chain of the amino acid.

4. The method of claim 1 wherein the nucleic acid-derived polypeptide comprises a T-helper epitope.

5. The method of claim 1 wherein the nucleic acid-derived polypeptide comprises a cytotoxic T lymphocyte (CTL) epitope.

6. The method of claim 5 wherein the CTL epitope is selected from the group list consisting of SEQ ID NOs:1, 2, 3, 4, 5 and 6.

7. The method of claim 1 wherein the nucleic acid-derived polypeptide comprises a B-cell epitope.

8. The method of claim 1 wherein the lipid or fatty acid is conjugated to a lysine, cysteine or serine residue.

9. The method of claim 1 wherein the lipid or fatty acid moiety is selected from the group list consisting of a palmitoyl, myristoyl, stearoyl and a decanoyl.

10. The method of claim 9 wherein the fatty acid moiety is lipoamino acid N-palmitoyl-S-[2,3-bis(palmitoyloxy)propyl]cysteine.

11. The method of claim 9 wherein the fatty acid moiety is S-[2,3-bis(palmitoyloxy)propyl]cysteine.

12. The method of claim 10, wherein the lipid moiety is a compound having a structure of general Formula (III): 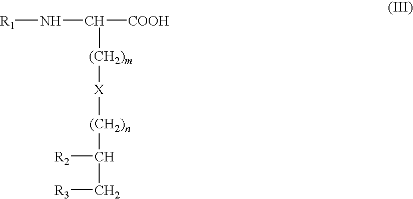
wherein:
(i) X is selected from the group consisting of sulfur, oxygen, disulfide (—S—S—), and methylene (—CH₂—), and amino (—NH—);
(ii) m is an integer being 1 or 2;
(iii) n is an integer from 0 to 5;
(iv) R₁ is selected from the group consisting of hydrogen, carbonyl (—CO—), and R′—CO—, wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group;
(v) R₂ is selected from the group consisting of R′—CO—O—, R₁—O—, R₁—O—CO—, R′—NH—CO—, and R′—CO—NH—, wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group; and
(vi) R₃ is selected from the group consisting of R′—CO—O—, R′—O—, R′—O—CO—, R′—NH—CO—, and R′—CO—NH—, wherein R′ is selected from the group consisting of alkyl having 7 to 25 carbon atoms, alkenyl having 7 to 25 carbon atoms, and alkynyl having 7 to 25 carbon atoms, wherein said alkyl, alkenyl or alkynyl group is optionally substituted by a hydroxyl, amino, oxo, acyl, or cycloalkyl group,
and wherein each of R₁, R₂ and R₃ is the same or different.

13. The method of claim 12, wherein X is sulfur; m and n are both 1; R₁ is selected from the group consisting of hydrogen, and R′—CO—, wherein R′ is an alkyl group having 7 to 25 carbon atoms; and R₂ and R₃ are selected from the group consisting of R′—CO—O—, R′—O—, R′—O—CO—, R′—NH—CO—, and R′—CO—NH—, wherein R′ is an alkyl group having 7 to 25 carbon atoms.

14. The method of claim 13, wherein R′ is selected from the group consisting of: palmitoyl, myristoyl, stearyl and decanol.

15. The method of claim 14, wherein R′ is palmitoyl.

16. The method of claim 12, wherein each integer R′ in said lipid moiety may be the same or different.

17. The method of claim 12, wherein X is sulfur; m and n are both 1; R₁ is hydrogen or R′—CO-wherein R′ is palmitoyl; and R₂ and R₃ are each R′—CO—O— wherein R′ is palmitoyl.

18. The method of claim 12, wherein the lipid moiety has the following General Formula (IV): 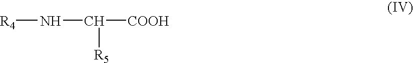
wherein:
R₄ is selected from the group consisting of:
(i) an alpha-acyl-fatty acid residue consisting of between about 7 and about 25 carbon atoms;
(ii) an alpha-alkyl-beta-hydroxy-fatty acid residue;
(iii) a beta-hydroxy ester of an alpha-alkyl-beta-hydroxy-fatty acid residue wherein the ester group is preferably a straight chain or branched chain comprising more than 8 carbon atoms; and
(iv) a lipoamino acid residue; and
R₅ is hydrogen or the side chain of an amino acid residue.

19. The method of claim 18, wherein R₄ consists of between about 10 and about 20 carbon atoms, and more preferably between about 14 and about 18 carbon atoms.

20. The method of claim 18, wherein R₄ is a lipoamino acid residue, so that the side-chain of the integers R₄ and R₅ can form a covalent linkage.

21. The method of claim 18, wherein the structure set forth in general Formula IV is a lipid moiety selected from the group consisting of: N,N′-diacyllysine; N,N′-diacylornithine; di(monoalkyl) amide or ester of glutamic acid; di(monoalkyl) amide or ester of aspartic acid; a N,O-diacyl derivative of serine, homoserine, or threonine; and a N,S-diacyl derivative of cysteine or homocysteine.

22. The method of claim 21, wherein the lipid moieties are further modified during synthesis or post-synthetically, by the addition of one or more spacer molecules.

23. The method of claim 21, wherein the lipid moieties are further modified during synthesis or post-synthetically, by the addition of one or more spacer molecules, wherein the spacer molecule is polyethylene glycol.

24. The method of claim 21, wherein the lipid moieties are further modified during synthesis or post-synthetically, by the addition of one or more spacer molecules, wherein the spacer molecule is polylysine.

25. The method of claim 21, wherein the lipid moieties are further modified during synthesis or post-synthetically by addition of one or more molecules carrying a functional group such as amino, sulphydryl, bromoacetyl, aminooxy group.

26. A vaccine comprising a nucleic acid-derived polypeptide conjugated to one or more lipid or fatty acid moieties to generate a self-adjuvanting immunogenic polypeptide which elicits an immune response across histocompatibility types.
